(19) United States
(12) Patent Application Publication
(10) Pub. No.: US 2011/0033706 A1
(43) Pub. Date: Feb. 10, 2011

(54) NANOCAPSULES AND METHODS FOR MODULAR ASSEMBLY

(76) Inventor: Yamuna Krishnan, Bangalore (IN)

Correspondence Address: FOLEY & LARDNER LLP, 150 EAST GILMAN STREET, P.O. BOX 1497, MADISON, WI, 53701-1497, US

(21) Appl. No.: 12/535,225

(22) Filed: Aug. 4, 2009

(51) Int. Cl. B32B 1/00; C07H 21/00; C07H 1/00

(52) U.S. Cl. 428/402; 536/22.1; 536/25.3; 977/700

(57) ABSTRACT

Disclosed are nanocapsules and methods of preparing these nanocapsules. The disclosure includes a modular assembly method of forming DNA nanocapsules. Discrete polyhedra are combined in a stepwise amalgamation to form icosahedra. The DNA nanocapsules may be used to encapsulate agents for drug delivery and other applications.

### TECHNICAL FIELD

This disclosure relates generally to nucleic acid nanostructures and methods for making the same. The nanostructures include nucleic acid-based nanoscapsules which may be used to encapsulate various substances.

### BACKGROUND

The following description is provided to assist the understanding of the reader. None of the information provided or references cited is admitted to be prior art to the present invention.

Nucleic acids are increasingly used as smart materials to construct patterned structures. For example, DNA is an extraordinarily versatile material for designing nano-architectural motifs, due in large part to its programmable G-C and A-T base pairing into secondary structures. These encoded structures are complemented by a sophisticated array of tools developed for DNA biotechnology: DNA can be manipulated using commercially available enzymes for site-selective DNA cleavage (restriction), ligation, labeling, transcription, amplification, replication, and methylation. DNA nanotechnology is further empowered by well-established methods for purification, structural characterization, and by solid-phase synthesis, so that designer DNA strands can be constructed.

Self-assembling nucleic acid structures represent a versatile system for nanoscale construction. Structure formation using nucleic acids begins with the chemical synthesis of single-stranded polynucleotides, which when properly annealed, self-assemble into nucleic acid tile building blocks through Watson-Crick base pairing. Nanofabrication through molecular self-assembly has resulted in the formation of DNA polyhedra with connectivities of cubes, tetrahedra, octohedra, dodecahedra, and buckminsterfullerene. These structures may lead to potential applications including nanoelectronics, nanomechanical devices, biosensors, programmable/autonomous molecular machines, and molecular computing systems. The programmed self-assembly of nucleic acid-based structures is a major challenge in nanotechnology and has numerous potential applications for nanofabrication of complex structures and useful devices.

### SUMMARY

The compositions and methods described herein relate to DNA-based platonic solids that may be obtained from degenerate components.

In one aspect, the disclosure provides an iscosahedral nanocapsule comprising: (a) one or more first five-way-junction (5WJ) modules each having a star-like configuration, wherein each first 5WJ module comprises five single-stranded oligonucleotides; (b) one or more second 5WJ modules each having a star-like configuration, wherein each second 5WJ module comprises five single-stranded oligonucleotides; and (c) one or more third 5WJ modules each having a star-like configuration, wherein each third 5WJ module comprises five single-stranded oligonucleotides; wherein the first 5WJ modules, the second 5WJ modules, and the third 5WJ modules are configured to interact to form an icosahedral nanocapsule. In an illustrative embodiment, the nanocapsule has two first 5WJ modules, five second 5WJ modules, and five third 5WJ modules.

In one embodiment, the five-single stranded oligonucleotides in each of the first, second, and third 5WJ modules are present in equimolar amounts. In one embodiment, each of the five single-stranded oligonucleotides in each of the first, second, and third 5WJ modules is configured to hybridize in an antiparallel orientation to another of the oligonucleotides over a portion of its length to form a partially double-stranded structure with five cohesive ends.

In one embodiment, one of the cohesive ends of each of the second 5WJ modules is configured to specifically hybridize with the five cohesive ends of a first 5WJ module and two complementary cohesive ends of adjacent 5WJ modules are configured to specifically hybridize; one of the cohesive ends of each of the third 5WJ modules is configured to specifically hybridize with the five cohesive ends of a first 5WJ module and two complementary cohesive ends of adjacent third 5WJ modules are configured to specifically hybridize; and two cohesive ends in each second 5WJ module are configured to specifically hybridize with two complementary cohesive ends in each third 5WJ module to form the DNA nanocapsule.

In one embodiment, each of the oligonucleotides is from 10 to 250 nucleotides in length. In one embodiment, each of the oligonucleotides is from 20 to 35 nucleotides in length. In one embodiment, each of the oligonucleotides is from 24 to 28 nucleotides in length. In one embodiment, the cohesive ends are from 4 to 16 nucleotides in length. In one embodiment, the cohesive ends are about 10 nucleotides in length. In one embodiment, one or more of the oligonucleotides in one or more of the first, second or third 5WJ modules comprises one or more unpaired bases at the vertex of the star-like configuration.

In one embodiment, the oligonucleotides are ligated. In one embodiment, the oligonucleotides are chemically ligated.

In one embodiment, the icosahedral nanocapsule has a tip-to-tip distance of 8 to 200 nm and a side-to-side distance of 8 to 200 nm. In one embodiment, the icosahedral nanocapsule has a tip-to-tip distance of 15 to 40 nm and a side-to-side distance of 15 to 40 nm. In one embodiment, the icosahedral nanocapsule has a pore size from 0.5 to 10 nm. In one embodiment, the icosahedral nanocapsule has a pore size from 1 to 5 nm.

In one embodiment, each edge of the icosahedral nanocapsule has a length from about 8 to 150 nucleotides. In one embodiment, each edge of the icosahedral nanocapsule has a length from about 20 to 30 nucleotides. In one embodiment, each edge of the icosahedral nanocapsule has a length of about 26 nucleotides. In one embodiment, each edge of the icosahedral nanocapsule has a length from about 3.4 to 50 nm. In one embodiment, each edge of the icosahedron has a length of about 8.0 to 10 nm.

In one embodiment, the nanocapsule has an internal encapsulation volume from 100 nm³ to 2.5×10⁵ nm³. In one embodiment, the nanocapsule has an internal encapsulation volume of at least 400 nm³.

In an illustrative embodiment, the first 5WJ module comprises oligonucleotides of SEQ ID NOs: 1-5, the second 5WJ module comprises oligonucleotides of SEQ ID NOs: 6-10, and the third 5WJ module comprises oligonucleotides of SEQ ID NOs: 11-15.

In one embodiment, the nanocapsule further comprises one or more agents encapsulated in the nanocapsule. In one embodiment, the agent is a biologically active agent. In one embodiment, the agent is a therapeutic agent or an imaging agent. In illustrative embodiments, the therapeutic agent is selected from the group consisting of an immunogenic peptide or protein, a chemotherapeutic agent, a toxin, a radiotherapeutic agent, a radiosensitizing agent, an antibiotic, and combinations thereof. In illustrative embodiments, the imaging agent is selected from the group consisting of paramagnetic, radioactive and fluorogenic chemical species.

In one embodiment, the nanocapsule further comprises a tagging moiety for linking the nanocapsule to other biomolecules. In one embodiment, the nanocapsule further comprises a labeling molecule. In one embodiment, the nanocapsule further comprises one or more of a targeting moiety, a fusogenic peptide, a membrane-permeabilizing peptide, a sub-cellular localization sequence, or a cell-receptor ligand. In one embodiment, the sub-cellular localization sequence targets the nanocapsule to a region of the cell selected from the group consisting of: the cytosol, the endoplasmic reticulum, the mitochondrial matrix, the chloroplast lumen, the medial trans-Golgi cisternae, the lumen of lysosome, and the lumen of an endosome.

In one aspect, the disclosure provides a method for assembling an icosahedral nanocapsule, the method comprising: (a) combining one or more first 5WJ modules with one or more second 5WJ modules to form a first six vertex structure, wherein each 5WJ module has a star-like configuration and comprises five single-stranded oligonucleotides; (b) combining one or more third 5WJ modules with one or more fourth 5WJ modules to form a second six vertex structure, wherein each 5WJ module has a star-like configuration and comprises five single-stranded oligonucleotides, and wherein the first 5WJ module and the third 5WJ module may be the same or different; and (c) combining the first and second six vertex structures to form an icosahedral nanocapsule. In one embodiment, steps (a), (b), and (c) occur sequentially. In one embodiment, steps (a), (b), and (c) do not occur simultaneously.

In one embodiment, the icosahedral nanocapsule has one first 5WJ module, five second 5WJ modules, one third 5WJ module, and five fourth 5WJ modules. In one embodiment, combining the first 5WJ module with five second 5WJ modules to form a first six vertex structure is by specific hybridization of one of the cohesive ends of each of the second 5WJ modules with the five cohesive ends of a first 5WJ module and specific hybridization of two complementary cohesive ends on adjacent second 5WJ modules. In one embodiment, combining the third 5WJ module with five fourth 5WJ modules to form a second six vertex structure is by specific hybridization of one of the cohesive ends of each of the second 5WJ modules with the five cohesive ends of a third 5WJ module and specific hybridization of two complementary cohesive ends on adjacent second 5WJ modules. In one embodiment, combining the first and second six vertex structures is by specific hybridization of two cohesive ends in each second 5WJ module with two complementary cohesive ends in each fourth 5WJ module.

In one aspect, the invention provides, a method for encapsulating an agent in a DNA nanocapsule, the method comprising: combining a first and second six vertex structure in the presence of one or more agents to form an icosahedral nanocapsule containing the one or more agents. In one embodiment, the first six vertex structure comprises a first 5WJ module and five second 5WJ modules, wherein each 5WJ module has a star-like configuration and comprises five single-stranded oligonucleotides; and wherein the second six vertex structure comprises a third 5WJ module and five fourth 5WJ modules, wherein each 5WJ module has a star-like configuration and comprises five single-stranded oligonucleotides, and wherein the first 5WJ module and the third 5WJ module may be the same or different.

In one aspect, the disclosure provides a half-icosahedral structure for the modular assembly of icosahedral nanocapsules, the structure comprising: (a) one first 5WJ module having a star-like configuration, wherein each first 5WJ module comprises five single-stranded oligonucleotides; (b) five second 5WJ modules each having a star-like configuration, wherein each second 5WJ module comprises five single-stranded oligonucleotides; wherein the first 5WJ module and the second 5WJ modules are configured to interact to form a half-icosahedral structure.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF FIGURES

FIGS. 1A, 1B, and 1C show an illustrative embodiment of a retrosynthetic strategy for the construction of the DNA icosahedron. The icosahedron is constructed from two half-icosahedra, VU₅ and VL₅, which in turn, are formed from two types of 5WJ, V and U/L.

FIGS. 2A, 2B, 2C, and 2D provide a series of images showing an illustrative embodiment of the formation of the complexes at various stages of assembly.

FIGS. 3A, 3B, 3C, and 3D provide a series of images showing an illustrative embodiment of the formation and characterization of [2:5:5] complexes.

FIG. 4A is a TEM image of an illustrative embodiment showing gold nanoparticles encapsulated within DNA icosahedra. The bright-field low-resolution TEM image shows the dense core of metallic particles within the cages. The inset shows representative high-resolution images in which the individual gold nanoparticles can be seen to be present within the icosahedral cages. Scale bar: 50 nm. FIG. 4B and FIG. 4C are graphs of histograms showing the size-based distribution of the gold nanoparticles before (B) and after encapsulation (C).

FIG. 5 shows an illustrative embodiment of the hybridization of the oligonucleotides in forming illustrative 5WJ structures.

FIG. 6 provides a gel electrophoresis image showing an illustrative embodiment of the formation of 5WJ from SS oligonucleotides: 15% Native polyacrylamide gel showing the formation of 5WJ from individual oligos. Lane 1. V1 oligo; Lane 2. V12; Lane 3. V123; Lane 4. V1234; Lane 5. V 5WJ.

FIG. 7 provides a gel electrophoretic image showing an illustrative embodiment of the formation of the [1:5] complex, VU₅ in the indicated stoichiometry. Radiolabeled V 5WJ was complexed with different ratios of unlabeled U 5WJ and samples were electrophoresed on 10% native PAGE in TBE buffer and visualized using PhosphorImager. Lane 1. ³²P labeled V1; Lane 2. V 5WJ; Lane 3. ³²P labeled Standard; Lane 4. V+U; Lane 5. V+2U; Lane 6. V+3U; Lane 7. V+4U, Lane 8. V+5U.

FIGS. 8A, 8B, and 8C show representative high resolution TEM images of [1:5] complexes (VU₅) using gold nanoparticle-functionalized U 5WJs. FIG. 8D is an illustrative nanocapsule assembly.

FIG. 9A provides a gel electrophoretic image of an illustrative embodiment of exonuclease treatment of the ligated [1:5] complex run on 0.8% Agarose. Lane 1. ligated [1:5] without treatment with exonuclease, Lane 2: ligated [1:5] treated with 5U of T7 exonuclease for 3 h at 25° C., Lane 3: ligated [1:5] complex treated with 3 U of Lambda exonuclease for 4 h at 37° C. FIG. 9B is a gel electrophoretic image of exonuclease treatment of the ligated [2:5:5] complex run on 0.8% Agarose gel. Lane 1. 0.5-12 kb DNA marker; Lane 2. Genomic DNA from Hela cells not treated with exonuclease; Lane 3. Genomic DNA treated with 3U of Lambda exonuclease for 4 h at 37° C.; Lane 4. Genomic DNA treated with 5U of T7 exonuclease for 3 h at 25° C.; Lane 5. Ligated [2:5:5] not treated with Lambda exonuclease, Lane 6. Ligated [2:5:5] treated with 3U of Lambda exonuclease for 4 h at 37° C.; Lane 7. Ligated [2:5:5] not treated with T7 exonuclease; Lane 8. Ligated [2:5:5] treated with 5U of T7 exonuclease for 3 h at 25° C.

FIG. 10 provides a series of size exclusion chromatograms of (A) [2:5:5] complex which was not treated with Lambda exonuclease, Rₜ=4.3 min; (B) [2:5:5] treated with 3U of Lambda exonuclease for 4 h, Rₜ=4.35 min; (C) Genomic DNA not treated with Lambda exonuclease, Rₜ=4.2 and 7.8 min; (D) Genomic DNA treated with 3U of Lambda exonuclease for 4 h. Inset: magnified trace of the same SEC profile; (E) [1:5] complex not treated with Lambda exonuclease, Rₜ=4.7 and 8.3 min; (F) [1:5] treated with 3U of Lambda exonuclease for 4 h, Rₜ=8.8 min.

FIG. 11 provides a graph of a histogram of DNA particle heights as measured by tapping mode Atomic Force Microscopy (AFM) on a sample size of 130 of 80 nM concentration of [2:5:5] complex, I in 100 mM phosphate buffer, pH 7.

FIG. 12 provides a series of transmission electron micrographs of an illustrative embodiment of ligated [2:5:5] complex. FIG. 12A is an image taken on Tecnai F20 FEI machine, operating voltage 200 kV, magnification-100 kX, Scale bar: 100 nm. FIG. 12B is an image taken on JEOL 100 CX II machine, operating voltage 80 kV, magnification 50 k X, Scale bar: 100 nm FIG. 12C is an image taken on Tecnai 12 Biotwin, operating voltage 100 kV, magnification 135 kX, Scale bar: 100 nm. Image has been intentionally defocused to obtain the edges of the particles formed due to Fresnel diffraction which indicates that this shape is due to the underlying structured particle and not an artifact of uranyl acetate staining inhomogeneity.

FIG. 13 shows a representation of (I) a basic triangular unit constructed using the overlapping cylinder model Icosahedron Modeling in Matlab 7; (II) a view of the resulting icosahedron showing the C-3 axis of symmetry; and (III) a view of the resulting icosahedron showing the C-5 axis of symmetry and cevian length, w.

FIG. 14 shows a representation of (I) an equilateral triangle with non-overlapping cylinders; and (II) a view of the resulting icosahedron showing the C-3 axis of symmetry.

FIG. 15 shows a TEM of an illustrative embodiment of gold nanoparticle (GNP) encapsulation.

FIG. 16 provides a series of TEM images of an illustrative embodiment of [2:5:5] complexes encapsulated with gold nanoparticles (GNP). FIG. 16A shows images of GNP-containing [2:5:5] complexes stained with uranyl acetate taken on JEOL 100 CX II machine, operating voltage 80 kV, magnification 50 kX, Scale Bar: 50 nm. FIG. 16B shows representative enlarged TEM images of individual [2:5:5] complexes with encapsulated gold nanoparticles showing C3 and C5 axes of symmetries characteristic of icosahedral geometry (n=50). Scale Bar: 20 nm. Bottom right hand image at the corner alone is taken at high resolution. FIG. 16C shows focused and defocused EM images of uranyl acetate-stained GNP-containing [2:5:5] complexes.

FIG. 17 provides a schematic showing the formation of an illustrative embodiment of [1:5:5] from [1:5] complex, VU₅ and lower 5WJ, L. The histograms show gold nanoparticle size distribution before (A) and after (B) encapsulation in [1:5:5] complex. FIG. 17C is a graph showing percentage change in gold nanoparticles sizes after encapsulation. Inset: Transmission electron micrograph of typical gold nanocluster in [1:5:5]. Scale bar: 20 nm.

FIG. 18 is an illustration showing putative orientations of the arms of the L 5WJ in the [1:5] and [1:5:5] complexes.

FIG. 19A is a photograph of a gel showing the formation of [2:5:5] complex from two complementary [1:5] complexes assembled from radiolabeled V 5WJ. Samples were electrophoresed on 0.8% Agarose in TAE buffer and visualized using PhosphorImager. Lane 1. ligated radiolabeled VU₅; Lane 2. ligated radiolabeled VL₅; Lane 3. Ligated [2:5:5] formed by complexation of radiolabeled VU₅ and unlabeled VL₅; Lane 4. Ligated [2:5:5] formed by complexation of radiolabeled VL₅ and unlabeled VU₅. FIGS. 19B and 19C are photographs of the same gel at higher contrast to show that there are no detectable intermediates.

FIG. 20 is a size exclusion chromatogram of the 2 MDa blue dextran, Rt=3.8 min.

FIG. 21 is a size exclusion chromatogram of dextran and the [2:5:5] complex.

### DETAILED DESCRIPTION

In the following detailed description, reference may be made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology is described herein using several definitions, as set forth throughout the specification. As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, the term “cohesive end” refers to single stranded nucleic acid (DNA or RNA) overhangs which are complementary to another DNA molecule, i.e. a paired cohesive end. These overhangs, also known as “sticky ends”, can be ligated to the complementary ends of another DNA molecule, using T4 DNA ligase, or other means. Different DNA fragments that can be ligated together by virtue of their sticky ends are said to possess compatible cohesive ends. In some embodiments, the single stranded overhangs of the cohesive ends may be at least 5, at least 10, at least 15, at least 20, or at least 30 nucleotides in length, but typically not more than about 50 nucleotides in length.

As used herein, the term “five-way-junction”, abbreviated “5WJ” refers to the convergence of complementary or partially complementary nucleic acid strands to form a star-like structure with five points.

As used herein, the term “icosahedron” refers to a convex regular polyhedron with twenty triangular faces, with five meeting at each of the twelve vertices. It has 30 edges and 12 vertices.

As used herein, the term “encapsulation” or “encapsulating” refers to the retention of substance within a compartment, delineated by a physical barrier. For example, the encapsulated agents described herein refer to agents which are retained within, and surrounded by a physical barrier, such as a nanocapsule.

As used herein, the term “nanocapsule” refers to a particle having a hollow core that is surrounded by a shell, such that the particle has a size of less than about 1,000 nanometers. When a nanocapsule includes an agent, the agent is typically located in the core that is surrounded by the shell of the nanocapsule.

As used herein, “nucleic acid,” “nucleotide sequence,” or “nucleic acid sequence” refer to a nucleotide, oligonucleotide, polynucleotide, or any fragment thereof and to naturally occurring or synthetic molecules. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

As used herein, an “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group. An oligonucleotide is a nucleic acid that includes at least two nucleotides.

One nucleic acid sequence may be “complementary” to a second nucleic acid sequence. As used herein, the terms “complementary” or “complementarity,” when used in reference to nucleic acids (i.e., a sequence of nucleotides such as an oligonucleotide), refer to sequences that are related by base-pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

Oligonucleotides as described herein may be capable of forming hydrogen bonds with oligonucleotides having a complementary base sequence. These bases may include the natural bases such as A, G, C, T and U, as well as artificial bases. An oligonucleotide may include nucleotide substitutions. For example, an artificial or modified base may be used in place of a natural base such that the artificial base exhibits a specific interaction that is similar to the natural base.

An oligonucleotide that is complementary to another nucleic acid will “hybridize” to the nucleic acid under suitable conditions. As used herein, “hybridization” or “hybridizing” refers to the process by which a oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. “Hybridizing” sequences which bind under conditions of low stringency are those which bind under non-stringent conditions (6×SSC/50% formamide at room temperature) and remain bound when washed under conditions of low stringency (2×SSC, 42° C.). Hybridizing under high stringency refers to the above conditions in which washing is performed at 2×SSC, 65° C. (where SSC is 0.15 M NaCl, 0.015 M sodium citrate, pH 7.2).

As used herein, the term “pore size” refers to the minimum size of substances that will be retained by a nanocapsule. In the context of some embodiments, the pore size of the nanocapsule must be selected so that the boundary is impermeable to the agent of interest. Thus, a nanocapsule with a nominal pore size of 2 nm means that particles greater than about 2 nm in diameter will be retained by the nanocapsule.

The term “localization sequence”, as used herein, refers to a molecule capable of recognizing a target cell or subcellular component thereof. Recognized subcellular components include the nucleus, ribosomes, mitochondria, and chloroplasts. Particular subcellular-localization components include the “nuclear-localization sequence” that aid in carrying molecules into the nucleus and are known to include the nuclear localization peptides and amino acid sequences.

The term “targeting moiety” means a moiety bound covalently or non-covalently to a nanocapsule, which moiety enhances the concentration of the nanocapsule in a target tissue relative to surrounding tissue.

As used herein, the term “therapeutic agent” refers to a compound that, when present in an effective amount, produces a desired therapeutic effect on a subject in need thereof.

### Nanocapsules

The compositions and methods described herein relate to nanocapsules having a modular structure, their production, and their use as research tools, including screening assays, as well as for diagnostic and therapeutic purposes among others.

The construction of well-defined 3D architectures is one of the greatest challenges of self-assembly. It is envisioned that DNA polyhedra can function as nanocapsules and thereby enable the targeted delivery of entities encapsulated from solution, for example. Key to realizing this envisaged function is the construction of complex polyhedra that provide a high encapsulation volume while preserving small pore size. Polyhedra based on platonic solids are particularly advantageous, as they maximize encapsulation volumes. In one aspect, the most complex DNA-based platonic solid, namely, an icosahedron, is provided through a unique modular assembly strategy.

One advantage of a modular assembly strategy described herein is that stoichiometrically well-defined closed capsules may be formed in high yield even at high concentrations as a result of the formation of prefolded intermediate modules as well as the increase in cooperativity associated with progressively higher order assembly. A prefolded scaffold with a defined curvature can undergo a larger number of favorable collisions that result in successful recognition events as a result of the degeneracy in the recognition sites that is associated with very few repeating modules. By contrast, nucleic acid tiles that have an identical structure form 2D sheets or large, irregular assemblies when combined at concentrations greater than about 20 nM. The present methods provide for the assembly of well-defined icosahedra at high concentrations of pre-defined modules (5WJs). In some embodiments, the 5WJs are present at a concentration of at least 5 μM, at least 10 μM, at least 20 μM, at least 30 μM or at least 50 μM. As such, the present methods provide for the assembly of relatively larger quantities of icosahedra than would otherwise be possible without the modular design.

In one aspect, the disclosure provides nanocapsules assembled from a plurality of different modules, termed “five-way-junctions” or “5WJs”. In one embodiment, the nanocapsule is assembled from at least two, at least three, at least four, at least five, or at least six different 5WJ modules. In an illustrative embodiment, the nanocapsule has two first 5WJ modules, five second 5WJ modules, and five third 5WJ modules.

Each 5WJ module may be constructed from a plurality of oligonucleotides to have a star-like configuration. Moreover, each 5WJ module may have cohesive ends which are designed to interact (via complementary base pairing) with cohesive ends of another 5WJ module. In one embodiment, each 5WJ module is constructed from at least three, at least four, or at least five single-stranded oligonucleotides. In one embodiment, each 5WJ module is constructed from equimolar amounts of five single-stranded oligonucleotides, which hybridize in an antiparallel orientation to another of the oligonucleotides over a portion of its length to form a partially double-stranded structure with five cohesive ends.

In one embodiment, each of the oligonucleotides is from 10 to 250 nucleotides in length. In one embodiment, each of the oligonucleotides is from 20 to 35 nucleotides in length. In one embodiment, each of the oligonucleotides is from 24 to 28 nucleotides in length. In an illustrative embodiment, one 5WJ module comprises oligonucleotides of SEQ ID NOs: 1-5, one 5WJ module comprises oligonucleotides of SEQ ID NOs: 6-10, and one 5WJ module comprises oligonucleotides of SEQ ID NOs: 11-15 (See Table 1).

In an illustrative embodiment, each of the 5WJ modules has cohesive ends at the terminus of each point in the star-like structure. For example, the cohesive end may be a single-stranded nucleic acid overhang, which is configured to specifically hybridize with a cohesive end of another 5WJ module. In one embodiment, the cohesive ends are from 4 to 16 nucleotides in length. In one embodiment, the cohesive ends are about 10 nucleotides in length. The length and base composition of the cohesive ends determines the specificity of the interaction. In general, the longer the overhang, the greater the specificity. The sequence of the overhang is not critical. One of skill in the art is able to design oligonucleotides that have complementary sequences.

In one embodiment, one or more of the oligonucleotides the 5WJ modules includes one or more unpaired bases at the vertex of the star-like configuration. The one or more unpaired bases at the vertices may permit flexibility during the formation of the nanocapsule. In particular embodiments, there are one, two, three, four, or five unpaired bases at the vertices of each 5WJ module.

The size and shape of the nanocapsule is determined by the length of each edge of the icosahedron. In turn, the length of each edge is determined by the length of the oligonucleotides that constitute the 5WJs. In one embodiment, each edge of the icosahedral nanocapsule has a length from about 8 to 150 nucleotides. In one embodiment, each edge of the icosahedral nanocapsule has a length from about 20 to 30 nucleotides. In one embodiment, each edge of the icosahedral nanocapsule has a length of about 26 nucleotides. In one embodiment, each edge of the icosahedral nanocapsule has a length from about 3.4 to 50 nm. In one embodiment, each edge of the icosahedron has a length of about 8.0 to 10 nm.

In one embodiment, the icosahedral nanocapsule has a tip-to-tip distance of 8 to 200 nm and a side-to-side distance of 8 to 200 nm. In one embodiment, the icosahedral nanocapsule has a tip-to-tip distance of 15 to 40 nm and a side-to-side distance of 15 to 40 nm. In one embodiment, the icosahedral nanocapsule has a pore size from 0.5 to 10 nm. In one embodiment, the icosahedral nanocapsule has a pore size from 1 to 5 nm. In one embodiment, the nanocapsule has an internal encapsulation volume from 100 nm³ to 2.5×10⁵ nm³. In one embodiment, the nanocapsule has an internal encapsulation volume of at least 400 nm³.

In one embodiment, the nanocapsules are resistant to exonuclease digestion, e.g., degradation by T7 and lambda exonucleases. Resistance to exonuclease digestion indicates the presence of circularized component DNA strands and is consistent with a highly catenated nature of the tertiary structure adopted by the nanocapsules.

The modular strategies described in the present methods may be used to construct icosahedra of even greater complexity through applications of quasiequivalence. The term “quasi-equivalence” means the relaxation of symmetry of regular polygons. Caspar and Klug (Cold Spring Harbor Symp. Quant. Biol., 1962, 27:1-24) showed that closed icosahedral shells can be constructed from pentamers and hexamers by minimizing the number T of nonequivalent locations that subunits occupy, with the T-number adopting the particular integer values 1, 3, 4, 7, 12, 13, . . . (T=h2+k2+hk, with h, k equal to non-negative integers). Quasiequivalence occurs in viral assembly when identical protein chains form a capsid with T>1, in which case the environments around the identical chains are not exactly identical. More than sixty subunits cannot be arranged in an equivalent fashion in an icosahedron, namely some will experience a different neighboring environment than others. In a T=3 virus, such as poliovirus, there are 3 different subunits per 60 triangles. In one embodiment, quasiequivalent nanocapsules are assembled by combining 5WJ modules with different structures or functionalities. For example, the addition of a functional group (e.g., label or targeting moiety) to one or more 5WJ modules in a nanocapsule will result in the formation of a nanocapsule with quasiequivalence because not all of the positions on the icosahedron will be identical.

In some embodiments, the shape of the nanocapsules is designed to mimic viruses to exploit viral entry pathways into cells. Given the capacity of icosahedra to function as efficient nanocapsules, targeted cargo delivery may occur through the use of an icosahedral display of protein-binding sites on such scaffolds to create virus like protein-DNA complexes that could be recognized by viral-entry pathways. Furthermore, the construction of an icosahedron facilitates the formation of extended 3D frameworks owing to the well-known propensity of this polyhedron to pack.

### Modular Assembly of Nanocapsules

In one aspect, nanocapsules are produced from discrete modules, termed five-way-junction (5WJ) modules, which ensures maximum flexibility when the modules are combined to form complete nanostructures. For instance, six 5WJs may be combined to form a six vertex structure, which is a half-icosahedron. Likewise, two six vertex structures may be combined to from a twelve vertex icosahedron. This modular design is advantageous in that it allows step-wise assembly of the complete nanocapsule from the component parts. The controlled assembly allows two six vertex structures (half-icosahedrons) to be combined in the presence of an agent in order to encapsulate that agent inside the nanocapsule.

In some embodiments, assembly of nanocapsules proceeds by sequential, non-covalent addition of specific modular assembly units (5WJs) during an assembly cycle. Attachment of each 5WJ is, by design, mediated by the specific, non-covalent binding of one or more pre-designated oligonucleotides to a complementary oligonucleotide on a different 5WJ. The process is carried out in a fashion such that a very large number of identical assemblies are fabricated simultaneously.

In some embodiments, the modular assembly methods fabricate nanocapsules in which: (a) each 5WJ occupies a specific, predetermined location in the nanocapsule; (b) multiple nanocapsules are assembled simultaneously; and (c) all the nanocapsules are identical in architecture and assembly unit order. In some embodiments, mixtures of nanocapsules that differ by size, structure, and/or functional groups may be assembled by combining modules for each desired nanocapsule in a single mixture. Due to the specificity of interaction between the different modules, only the desired structures will be generated.

The size of the nanocapsule will be defined by the length of the individual edges, which, in turn, are defined by the lengths of the oligonucleotides that form the 5WJ modules. Each position in the nanocapsule can be uniquely defined through the process of modular assembly, and functional groups can be added at any desired position. This system enables manufacture of complex nanocapsules.

In one embodiment, a series of units are added in a given pre-designed order to an initial 5WJ module and/or nanocapsule intermediate. Additional 5WJ modules are added sequentially in a procedure akin to solid phase polymer synthesis. Addition of each 5WJ module to the nanocapsule intermediate undergoing assembly depends upon the nature of the cohesive ends of oligonucleotides presented by the previously added modules. Thus, modules can bind primarily to the cohesive ends of oligonucleotides exposed on the nanocapsule intermediate undergoing assembly.

This scheme provides for assembly of complex nanocapsules using relatively few non-cross-reacting, complementary joining pairs of nucleic acids. Nucleic acids have well-defined binding and recognition properties, and the technology to manipulate the intermolecular interactions of nucleic acids is well known in the art. Only a few cohesive ends need to be used, since only a limited number of cohesive ends will be exposed on the surface of an assembly intermediate at any one step in the assembly process. Modules with complementary cohesive ends can be added and incubated with the nanocapsule intermediate, causing the added modules to be attached to the nanocapsule intermediate during an assembly cycle.

FIG. 1 depicts an embodiment of the modular assembly method in one dimension. In step 1, three different types of 5WJ modules are formed from component oligonucleotides that have specific complementarity. In step 2, these 5WJ modules are joined to form two six vertex intermediates. In step 3, the two six vertex intermediates are joined together to form a twelve vertex icosahedron nanocapsule.

After each step in the method of modular assembly, excess unbound modules may be removed from the nanocapsule intermediate by a removal step, e.g., a washing step. The washing should be able to remove subunits held by non-specific interactions without disrupting the specific, interactions of complementary joining elements. Appropriate solvents may vary as to pH, salt concentration, chemical composition, etc., as required by the modules being used.

The above-described steps of adding modules can be repeated in an iterative manner until a complete nanocapsule is assembled. Modules may be added individually or, in certain embodiments, they can be added as subassemblies. The result is a completely defined nanocapsule that satisfies the desired design parameters.

The order in which modules are added is determined by the desired structure of the nanocapsule, and the need to minimize the number of cross-reacting cohesive end pairs used in the assembly process. Hence, determining the order of assembly is an integral part of the design of a nanocapsule to be fabricated by modular assembly. Cohesive ends of the oligonucleotides in each 5WJ module, by design, permit assembly of the desired nanostructure. The cohesive ends are mixed and matched as needed to fabricate modular units with the joining elements that will place the modules in the desired spatial orientation. Moreover, only a small number of complementary joining elements are required for the fabrication of a large number of unique and complex nanostructures, since only one type of assembly unit is added in each staged assembly cycle and, therefore, joining elements can be used repeatedly without rendering ambiguous the position of an assembly unit within the completed nanostructure.

Structural integrity of the nanostructure is of importance throughout the process of staged assembly, and the 5WJ modules are suitably connected by non-covalent interactions. A specific non-covalent interaction is, for example, specific hybridization between complementary oligonucleotides. The specific interaction should exhibit adequate affinity to confer stability to the complex between the 5WJ modules sufficient to maintain the interaction stably throughout the entire assembly process. A specific non-covalent interaction should exhibit adequate specificity such that the added assembly unit will form stable interactions only with joining modules designed to interact with it.

In certain embodiments, the fabrication of a nanocapsule by the modular assembly methods involves joining relatively rigid and stable 5WJ modules, using non-covalent interactions between and among modules. Therefore, in certain embodiments, individual assembly units may include flexible domains. In some embodiments, each 5WJ module has one or more unpaired bases at the vertices to permit flexibility during the formation of the nanocapsule. In particular embodiments, there are one, two, three, four, or five unpaired bases at the vertices of each 5WJ module.

In certain embodiments, nanocapsules fabricated according to the staged assembly methods disclosed herein are subsequently stabilized by chemical fixation or by cross-linking In some embodiments, a suitable agent may be used to stabilize the nanocapsule after it has been assembled. In one embodiment, the agent is capable of ligating the oligonucleotides of the 5WJ modules together by enzymatic or non-enzymatic chemical ligation. In a particular embodiment, the non-enzymatic chemical ligation is by treatment with N-cyano imidazole (NCI).

In some embodiments, the 5WJ modules or any intermediate may contain an added functional element, such as a label or a targeting moiety. In one embodiment, the nanocapsules is linked to a label. The label can be any material having a detectable physical or chemical property. Such detectable labels have been well-developed in the field of nucleic acid assays and imaging. The label can be coupled directly or indirectly to the desired component according to methods well known in the field. As indicated above, a wide variety of labels can be used, with the choice of label depending on the sensitivity required, ease of conjugation with the compound, stability requirements, available instrumentation, and disposal provisions. In one embodiment, the label is coupled indirectly via a biotinylated label bound to avidin which is coupled to the nanocapsule. In one embodiment, the oligonucleotides of the nanocapsule may be labeled with a fluorescent dye or a fluorophore. A fluorescent dye or a fluorophore is a chemical group that can be excited by light to emit fluorescence. Some fluorophores may be excited by light to emit phosphorescence.

In some embodiments, the nanocapsules are linked to a targeting moiety that directs the nanocapsule to a desired cell or cellular compartment. Targeting agents are compounds with a specific affinity for a target compound, such as a cell surface epitope associated with a specific disease state. Targeting agents may be attached to a nanocapsule surface to allow targeting of the nanocapsule to a specific target. Examples of targeting moieties that may be used to direct nanocapsules to cells include, but are not limited to, antibodies, antigen-binding antibody fragments, receptor-binding polypeptides, or receptor-binding polysaccharides. Other examples of targeting agents include an amino acid sequence, including the RGD peptide, an NGR peptide, folate, Transferrin, GM-CSF, Galactosamine, peptide linkers including growth factor receptors (e.g. IGF-1R, MET, EGFR), antibodies and antibody fragments including anti-VEGFR, Anti-ERBB2, Anti-tenascin, Anti-CEA, Anti-MUC1, Anti-TAG72, mutagenic bacterial strains, and fatty acids. The size of these targeting moieties varies widely, ranging from large antibodies to small growth factors, cytokines and antibody fragments.

Examples of targeting moieties that may be used to direct nanocapsules to cellular compartments include, but are not limited to, the amino terminal 81 amino acids of human type II membrane-anchored protein galactosyltransferase for directing the nanocapsule to the Golgi and the amino terminal 12 amino acids of the presequence of subunit IV of cytochrome c oxidase for directing a nanocapsule to the mitochondrial matrix. The 12 amino acids of the presequence of subunit IV of cytochrome c oxidase may be linked to the nanocapsule through a linker sequence.

### Therapeutic Compositions and Methods

In one aspect, the nanocapsules described herein may be used to encapsulate and deliver a wide variety of agents, including bioactive, diagnostic, and visualization agents to a cell or tissue. Agents include markers, visualization agents, fluorescent particles, adjuvants, dendritic cell maturation factors, and antigens, e.g. proteins foreign to the immune system receiving the proteins. Additional agents include, but are not limited to polynucleotides, polypeptides, genetic material, peptide nucleic acids, aptamers, carbohydrates, mini-chromosomes, molecular polymers, aggregates or associations of an inorganic or organic nature, genes, any other macromolecule or any combination of any of these. The agents may be released after being taken up by cells.

The agent to be encapsulated in the nanocapsule may be supplied as an individual agent or supplied in various prepared mixtures of two or more agents that are subsequently combined to form the agent.

Some non-exhaustive examples of agents that may be encapsulated include, but are not limited to, adrenergic, adrenocortical steroid, adrenocortical suppressant, aldosterone antagonist, and anabolic agents; analeptic, analgesic, anesthetic, anorectic, and anti-acne agents; anti-adrenergic, anti-allergic, anti-amebic, anti-anemic, and anti-anginal agents; anti-arthritic, anti-asthmatic, anti-atherosclerotic, antibacterial, and anticholinergic agents; anticoagulant, anticonvulsant, antidepressant, antidiabetic, and antidiarrheal agents; antidiuretic, anti-emetic, anti-epileptic, antifibrinolytic, and antifungal agent; antihemorrhagic, inflammatory, antimicrobial, antimigraine, and antimiotic agents; antimycotic, antinauseant, antineoplastic, antineutropenic, and antiparasitic agents; antiproliferative, antipsychotic, antirheumatic, antiseborrheic, and antisecretory agents; antispasmodic, antithrombotic, anti-ulcerative, antiviral, and appetite suppressant agents; blood glucose regulator, bone resorption inhibitor, bronchodilator, cardiovascular, and cholinergic agents; fluorescent, free oxygen radical scavenger, gastrointestinal motility effector, glucocorticoid, and hair growth stimulant agent; hemostatic, histamine H2 receptor antagonists; hormone; hypocholesterolemic, and hypoglycemic agents; hypolipidemic, hypotensive, and imaging agents, immunizing and agonist agents; mood regulators, mucolytic, mydriatic, or nasal decongestant; neuromuscular blocking agents; neuroprotective, NMDA antagonist, non-hormonal sterol derivative, plasminogen activator, and platelet activating factor antagonist agent; platelet aggregation inhibitor, psychotropic, radioactive, scabicide, and sclerosing agents; sedative, sedative-hypnotic, selective adenosine A1 antagonist, serotonin antagonist, and serotonin inhibitor agent; serotonin receptor antagonist, steroid, thyroid hormone, thyroid hormone, and thyroid inhibitor agent; thyromimetic, tranquilizer, amyotrophic lateral sclerosis, cerebral ischemia, and Paget's disease agent; unstable angina, vasoconstrictor, vasodilator, wound healing, and xanthine oxidase inhibitor agent; immunological agents, antigens from pathogens, such as viruses, bacteria, fungi and parasites, optionally in the form of whole inactivated organisms, peptides, proteins, glycoproteins, carbohydrates, or combinations thereof, any examples of pharmacological or immunological agents that fall within the above-mentioned categories and that have been approved for human use that may be found in the published literature, any other bioactive component, or any combination of any of these.

In one embodiment, nanocapsules may be used to deliver antigenic proteins, e.g., non-native proteins, proteoglycans, polysaccharides, or nucleic acids. Antigenic proteins are proteins that evoke a humoral immune response from an immune system to which they are delivered. For some applications, the nanocapsules deliver the biological agent to antigen presenting cells (APCs). The antigenic function of the protein may be used in the context of vaccines and immune system research tools. APCs, e.g., dendritic cells, that receive nanocapsules process the agents in the nanoparticles and present antigenic portions to other immune system cells, so that the immune system in a body generates antibodies against the antigen, and to other agents that also express the antigen.

In one embodiment, nanocapsules may be used to deliver nucleic acids to cells, e.g., siRNA molecules or expression vectors. For instance, nucleic acids can be incorporated into vectors, and the vectors may be encapsulated within nanocapsules. As used herein, a “vector” is a replicon, such as a plasmid, phage, or cosmid, into which another nucleic acid segment may be inserted so as to bring about replication of the inserted segment. Vectors typically are expression vectors containing an inserted nucleic acid segment that is operably linked to expression control sequences. An expression vector is a vector that includes one or more expression control sequences, and an expression control sequence is a DNA sequence that controls and regulates the transcription and/or translation of another DNA sequence. Expression control sequences include, for example, promoter sequences, transcriptional enhancer elements, and any other nucleic acid elements required for RNA polymerase binding, initiation, or termination of transcription. With respect to expression control sequences, “operably linked” means that the expression control sequence and the inserted nucleic acid sequence of interest are positioned such that the inserted sequence is transcribed (e.g., when the vector is introduced into a host cell). For example, a DNA sequence is operably linked to an expression-control sequence, such as a promoter when the expression control sequence controls and regulates the transcription and translation of that DNA sequence. Examples of vectors include: plasmids, adenovirus, Adeno-Associated Virus (AAV), Lentivirus (Fly), Retrovirus (MoMLV), and transposons.

### Diagnostic Compositions and Methods

In one aspect, nanocapsules may be used to encapsulate diagnostic or visualization agents. Visualization agents are materials that allow the nanoparticles to be visualized after exposure to a cell or tissue. Visualization includes imaging for the naked eye, as well as imaging that requires detecting with instruments or detecting information not normally visible to the eye, and includes imaging that requires detecting of photons, sound or other energy quanta. Examples include stains, vital dyes, fluorescent markers, radioactive markers, enzymes or plasmid constructs encoding markers or enzymes. Many materials and methods for imaging and targeting that may be used in nanoparticles are provided in the Handbook of Targeted delivery of Imaging Agents, Torchilin, ed. (1995) CRC Press, Boca Raton, Fla.

Visualization based on molecular imaging typically involves detecting biological processes or biological molecules at a tissue, cell, or molecular level. Molecular imaging can be used to assess specific targets for gene therapies, cell-based therapies, and to visualize pathological conditions as a diagnostic or research tool. Imaging agents that are able to be delivered intracellularly are particularly useful because such agents can be used to assess intracellular activities or conditions. Imaging agents must reach their targets to be effective; thus, in some embodiments, an efficient uptake by cells is desirable.

Further, imaging agents should provide high signal to noise ratios so that they may be detected in small quantities, whether directly, or by effective amplification techniques that increase the signal associated with a particular target. Amplification strategies are reviewed in Allport and Weissleder, Experimental Hematology 1237-1246 (2001), and include, for example, avidin-biotin binding systems, trapping of converted ligands, probes that change physical behavior after being bound by a target, and taking advantage of relaxation rates. Examples of imaging technologies include magnetic resonance imaging, radionuclide imaging, computed tomography, ultrasound, and optical imaging.

Suitable imaging agents include, for example, fluorescent molecules, labeled antibodies, labeled avidin:biotin binding agents, colloidal metals (e.g., gold, silver), reporter enzymes (e.g., horseradish peroxidase), superparamagnetic transferrin, second reporter systems (e.g., tyrosinase), and paramagnetic chelates. Advantages of nanocapsules less than about 100 nm or 50 nm in diameter include for example, the ability of the nanoparticles to be readily delivered and taken up by cells.

Compared to imaging agents that are merely conjugated to a targeting molecule, nanocapsules can increase signal-to-noise ratio by delivering larger imaging agent loads per uptake event resulting in higher amplification. Many imaging agents may be loaded into a particle having a targeting molecule (e.g., tenascin), which passes into a cell via a single uptake event (i.e., caveolar uptake in the case of nanoparticles of less than about 100 nm or 50 nm). In contrast, only a single imaging agent linked to a targeting molecule would be taken up by the same event. Since the internalization, intracellular transport, and recycling of cell surface receptors often requires significant turnaround time, the resultant direct uptake of signal molecules by a cell is slower than the uptake of signal molecules with a nanoparticle.

Magnetic resonance imaging contrast agents may also be used in nanocapsules. Examples of Magnetic resonance imaging contrast agents include, but are not limited to, 1,4,7,10-tetraazacyclododecane-N,N′,N″N′″-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (Do3A) and derivatives thereof. X-Ray contrast agents may also be incorporated into nanoparticles, which may be delivered to a patient, tissue, or cell. X-ray contrast agents already known in the art include a number of halogenated derivatives, especially iodinated derivatives, of 5-amino-isophthalic acid.

Formulations of Pharmaceutical Compositions. The nanocapsule compositions described herein can be incorporated into pharmaceutical compositions suitable for administration. The pharmaceutical compositions generally comprise nanocapsules encapsulating one or more therapeutic or diagnostic agents and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the antibody compositions (see, e.g., Remington's Pharmaceutical Sciences, Mack Publishing Co., Easton, Pa. 18^{th} ed., 1990). The pharmaceutical compositions are generally formulated to be sterile, substantially isotonic, and in full compliance with all Good Manufacturing Practice (GMP) regulations of the U.S. Food and Drug Administration.

The terms “pharmaceutically-acceptable” and “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of aerosol compositions, gaseous.

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the nanoparticle compositions, use thereof in the compositions is contemplated. Supplementary active compounds can also be incorporated into the compositions.

A pharmaceutical composition is formulated to be compatible with its intended route of administration. The nanocapsule compositions can be administered by parenteral, topical, intravenous, oral, subcutaneous, intra-arterial, intradermal, transdermal, rectal, intracranial, intraperitoneal, intranasal, or intramuscular routes, or as inhalants. The nanocapsule compositions can be optionally administered in combination with other agents that are at least partly effective in treating various diseases.

Solutions or suspensions used for parenteral, intradermal, intranasal, or subcutaneous application can include the following components: a sterile diluent for injection such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes, or multiple-dose vials made of glass or plastic.

Pharmaceutical compositions suitable for injectable use include sterile aqueous solutions (where the composition is water soluble) or dispersions and sterile powders for the extemporaneous preparation of sterile injectable solutions or inhaling or dispersion. For intravenous administration, suitable carriers include physiological saline, bacteriostatic water, Cremophor ELTM (BASF, Parsippany, N.J.), or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion, and by the use of surfactants. Prevention of the action of microorganisms can be achieved by various antibacterial and antifungal compounds, e.g., parabens, chlorobutanol, phenol, ascorbic acid, thimerosal, and the like. In many cases, one may include isotonic compounds, e.g., sugars, polyalcohols such as manitol and sorbitol, or sodium chloride, in the composition.

Sterile, injectable solutions can be prepared by incorporating the nanocapsule compositions in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation include vacuum-drying and freeze-drying to yield a powder of the active ingredient, plus any additional desired ingredient, from a previously sterile-filtered solution thereof. The agents can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the binding agent can be incorporated with excipients and used in the form of a tablet, troche, or capsule. Oral compositions can also be prepared using a fluid carrier for use as a mouthwash, wherein the compound in the fluid carrier is applied orally and swished and expectorated or swallowed. Pharmaceutically compatible binding compounds and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

### Methods of Using Nanocapsules as Chemical Reactors

In one embodiment, the nanocapsules described herein may be used as nanoscale reaction vessels for studying the behavior or interaction of molecules in confinement. The method for the preparation of nanocapsules involves localization of reactants (atoms, ions or molecules) within a nanocapsule. For example, a nanocapsule may be assembled from six or twelve vertex structures in the presence of atoms, ions or molecules to produce a nanocapsule encapsulating the atoms, ions or molecules. The interaction of the atoms, ions or molecules within the nanoscapsule may be observed using microscopy, spectroscopy or other means.

### Kits

The materials and components described for use in the methods may be suited for the preparation of a kit. Thus, the disclosure provides a kit useful for encapsulating various agents, including therapeutic and diagnostic agents. For example, in one embodiment, the kit can comprise a one or more 5WJ modules for assembling an icosahedral nanocapsule. In another embodiment, the kit can comprise a six vertex structure (half-icosahedron), which may be assembled in the presence of an agent to produce a nanocapsule encapsulating the agent.

The oligonucleotides and 5WJ modules are easily synthesized and are stable in various formulations for long periods of time, particularly when lyophilized or otherwise dried to a powder form. In this form, they are easily reconstituted for use by those of skill in the art. Other reagents and consumables required for using the kit could be easily identified and procured by those of skill in the art who wish to use the kit. The kits can also include buffers useful in the methods of the technology. The kits may contain instructions for the use of the reagents and interpreting the results.

Diagnostic kits may also be prepared that comprise nanocapsules and suitable imaging agents, as well as, optionally, diagnostic tools for deliveries of the particles and agents, and instructions for a diagnostic use. For example, a kit may comprise nanocapsules that comprise imaging agents and ligands that are targeted to bind to target molecules. The nanocapsules can be administered to, for example, a patient, a body portion, a sample, a specimen, or a test apparatus. The targeting molecule, e.g., an antibody or ligand, becomes associated with a target molecule, e.g., to indicate its presence. Such kits are useful for clinical, medical, and research uses. For example, a kit may be made to image molecules present on a histology slide, or a thin section. The kit may further include instructions for use. Instructions may be, for example, an insert, a label, on packaging, a brochure, a handout, a pamphlet, a web page, or in written or electronic form, including posting on internet sites or intranet locations. Instructions may provide general information for use of the kit, and also provide, for example, information on targets, disease states, and/or targeting molecules. Target molecules may be used, for example, that indicate a disease state, a pathology, or a test result, and may provide qualitative or quantitative indicia.

For example, many peptides have been developed that are specific to certain molecules cell types, and/or tissue types; such peptides may be used a targeting molecules on nanoparticles to target the nanoparticles and their contents. A peptide is used broadly to mean a linear, cyclic or branched peptide, peptoid, peptidomimetic, or the like. Methods for identifying peptides suitable for targeting are also known, see e.g., U.S. Pat. Nos. 6,306,365, 6,296,832 and 6,232,287.

Also, nanoparticles may be made with antibodies that recognize antigens, including antigens that are diagnostic of a condition in a patient, body, sample, specimen, or test. Or ligands that bind to the antigens may be used. Antigens may thus be detected to determine if a particular molecule is present.

### EXAMPLES

The present methods and kits, thus generally described, will be understood more readily by reference to the following examples, which are provided by way of illustration and are not intended to be limiting of the present methods and kits. The following is a description of the materials and experimental procedures used in the Examples.

Oligonucleotides. All oligonucleotides were purchased from Sigma and quantified by their UV absorbance at 260 nm. The five arm junctions V, U and L (FIG. 1a), were made from five oligonucleotides each, the sequences of which are shown in Table 1.

TABLE 1
Oligonucleotide Sequences.

| | | SEQ ID |
|---|---|---|
| Name | Sequence (5′ to 3′) | NO: |
| V1 | GC CTG GTG CC ACC GGT GA CGT TCC GC | SEQ ID |
| | | NO: 1 |
| V2 | GC CTG GTG CC CCG CGT CC TCA CCG GT | SEQ ID |
| | | NO: 2 |
| V3 | GC CTG GTG CC GCC ACG CT TT GGA CGC GG | SEQ ID |
| | | NO: 3 |
| V4 | GC CTG GTG CC GCG AGT GC AA AGC GTG GC | SEQ ID |
| | | NO: 4 |
| V5 | GC CTG GTG CC GCG GAA CG AA GCA CTC GC | SEQ ID |
| | | NO: 5 |
| U1 | GG CAC CAG GC GCG GAA CG AA GCA CTC GC | SEQ ID |
| | | NO: 6 |
| U2 | GC GAC TGA TG GCG AGT GC AA AGC GTG GC | SEQ ID |
| | | NO: 7 |
| U3 | TT ATA GGA CT GCC ACG CT TT GGA CGC GG | SEQ ID |
| | | NO: 8 |
| U4 | TT ATA GGA CT CCG CGT CC TCA CCG GT | SEQ ID |
| | | NO: 9 |
| U5 | CA TCA GTC GC ACC GGT GA CGT TCC GC | SEQ ID |
| | | NO: 10 |
| L1 | GG CAC CAG GC GCG GAA CG AA GCA CTC GC | SEQ ID |
| | | NO: 11 |
| L2 | GC GAC TGA TG GCG AGT GC AA AGC GTG GC | SEQ ID |
| | | NO: 12 |
| L3 | AG TCC TAT AA GCC ACG CT TT GGA CGC GG | SEQ ID |
| | | NO: 13 |
| L4 | AG TCC TAT AA CCG CGT CC TCA CCG GT | SEQ ID |
| | | NO: 14 |
| L5 | CA TCA GTC GC ACC GGT GA CGT TCC GC | SEQ ID |
| | | NO: 15 |

Phosphorylation. The 5′ ends of oligonucleotides at 1 mM strand concentration were phosphorylated using T4 Polynucleotide Kinase (10 units, NEB). The reaction mixture containing oligonucleotides and dATP in the ratio of 1:3 was incubated at 37° C. for 2 h and then heated to 75° C. for 10 min to inactivate the protein. This was later subjected to ethanol precipitation and the precipitated DNA was quantified.

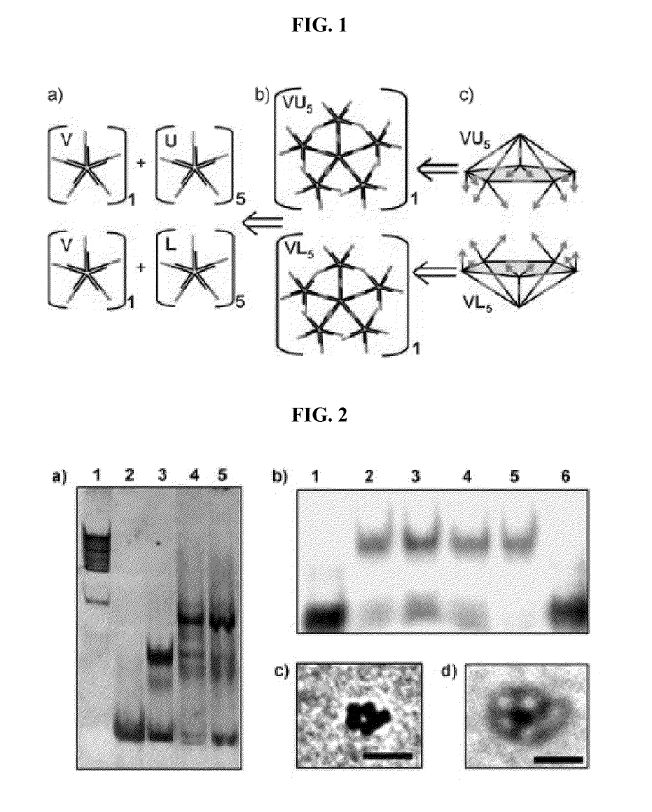

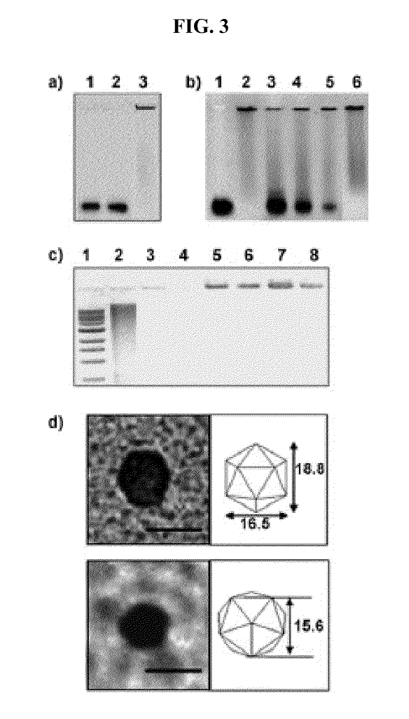

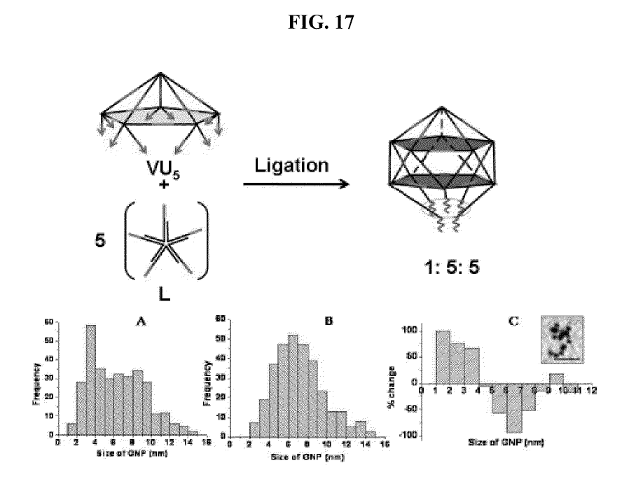

5WJs: V, U and L. Phosphorylated oligonucleotides, 20 μM, were mixed in equimolar quantities in sodium phosphate buffer (10 mM phosphate buffer, pH 6, 100 mM NaCl, 1 mM M902) and heated at 90° C. for 20 min and then annealed at the rate of 0.33° C./min to 20° C. The samples were incubated for 2 h at 20° C. and then equilibrated at 4° C. for 72 h to form 5WJs. The formation of 5WJs was ensured by 15% native PAGE (FIG. 2A & FIG. 6), in-gel yield, 62%.

[1:5] complexes: VU₅ and VL₅. A 1:5 ratio of V:U/L (20 VM) was mixed well and heated to 50° C. for 4 h and samples were annealed at the rate of 0.33° C./min to 20° C. The samples were incubated at 20° C. for 2 h and equilibrated at 4° C. for 72 h. (FIGS. 2B, 2C, & 7), in-gel yield, 73%. VU₅ and VL₅ were ligated chemically using N-Cyano Imidazole (NCI), prior to further complexation. For gold-nanoparticle labeled 1:5 complexes, the latter was made similarly using thiol labeled U 5WJ (5′ thiol modified U4 oligo) in buffer containing 2 mM DTT. To this, gold nanoparticles (3.5 nm, 200 nM) were added and incubated at 40° C. for 3 h followed by dialysis. The dialysed solution was visualized by TEM as previously described.

[2:5:5] complex, I. VU₅ and VL₅ (after chemical ligation) were mixed in equimolar ratios, heated to 50° C. for 4 h and annealed at the rate of 0.33° C./min to 20° C. The samples were incubated at 20° C. for 2 h and then equilibrated at 4° C. for 72 h. (FIG. 3A). The [2:5:5] complex was subjected to chemical ligation as described below.

[1:5:5] complex: VU₅L₅. 20 μL of VU₅ (4 μM) and 20 μL of L 5WJ (20 μM) were mixed in sodium phosphate buffer (10 mM phosphate buffer, pH 6, 100 mM NaCl, 1 mM, MgCl₂). The resultant solution was heated to 45° C. for 4 h and then annealed at a rate of 0.33° C./min up to 20° C. The sample was allowed to equilibrate at 4° C. for 72 h and ligated using NCI. For encapsulation, the reaction was carried out in the presence of gold nanoparticles of different sizes. After ligation, the resulting solution was dialyzed and the aliquot of this sample dried on a carbon coated copper grid, stained with uranyl acetate and viewed under bright field TEM (JEOL 100 CX II, 80 kV and Tecnai G12 Biotwin, 100 κV). The sizes of the gold nanoparticles present in clusters were measured and plotted for comparison (FIG. 17).

Chemical ligation. Non-enzymatic-chemical ligation was performed to ligate the contiguously positioned 3′ hydroxyl and 5′ phosphate groups. N-Cyano Imidazole (NCI, 50 mM) was used for ligation reaction. NiCl₂, 50 mM, was added to increase ligation efficiency. The cocktail was incubated at 24° C. for 24 h. NCI has been shown to ligate only 3′ and 5′ ends of 5′ phosphorylated oligonucleotides that are contiguously hybridized on a template. Post ligation, the samples were dialyzed against 10 mM phosphate buffer, pH 6.0, 100 mM NaCl, 1 mM MgCl₂.

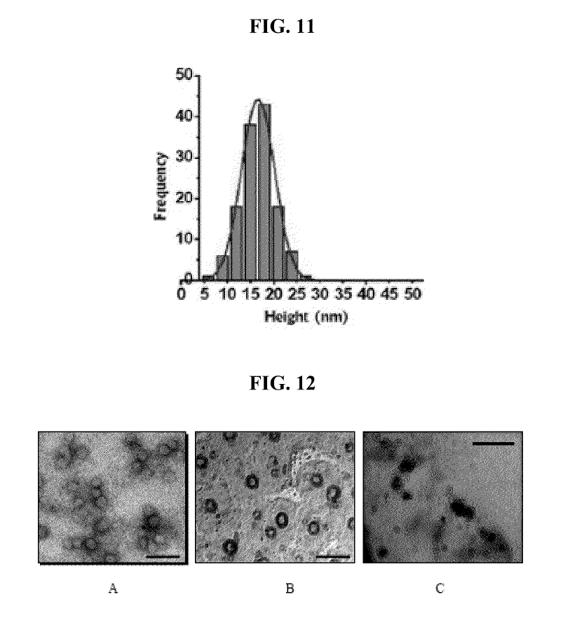

Atomic Force Microscopy (AFM). 3 μL of the sample (73 nM of [2:5:5] complex) was placed on a freshly cleaved mica surface and spread evenly. The mica surface was then washed three times with deionized water and air-dried for 15 min. The residual water was dried under a steady flow of Nitrogen. 5 μm×5 μm area of the mica was then imaged (WITec α-SNOM, in the tapping mode). Silicon Nitride (Molecular Imaging) cantilevers with spring constants of 42 N·m⁻¹ and resonant frequency of 320 KHz were used for intermittent contact imaging (Acoustic AC mode). Image-Ctrl was used for all image analysis. (FIG. 11).

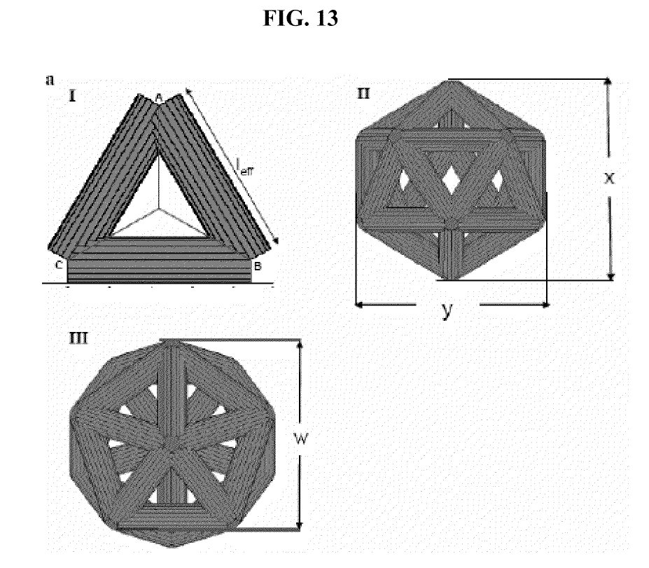

Transmission Electron Microscopy. Specimens were adsorbed on the carbon coated and glow discharged surface of a 400 mesh copper grid (Pelco and Ted Pella, USA) by flotation for 20 min, excess moisture blotted, stained using 1% uranyl acetate solution and imaged under 100/200 KV operating voltage of a transmission electron microscope (Tecnai 12 Biotwin/Tecnai F20, FEI Co. The Netherlands). Images were recorded at 1024×768 pixel resolution using an onboard side mounted retractable CCD camera (Megaview IIII, SIS, Germany) and analyzed using the I-TEM™ software. For Platinum shadowing, samples loaded on a Carbon Type-B, 400 mesh Copper grid were rotary platinum shadowed for 10 seconds in a vacuum evaporator at 0.8×10⁻⁴ Torr pressure and viewed under a bright-field TEM (JEOL 100 CX II) operating at an acceleration voltage of 80 kV, with the filament current in the range of 3.4 nA to 7 nA (FIG. 3D & FIG. 12). Cevian lengths, and other related dimensions of the C3 and C5 views of the icosahedral particles were obtained from pre-selected particles that clearly exhibited either the C3 or the C5 axes of symmetry indicating that (a) they were well-formed and (b) where there was no ambiguity in the distances measured.

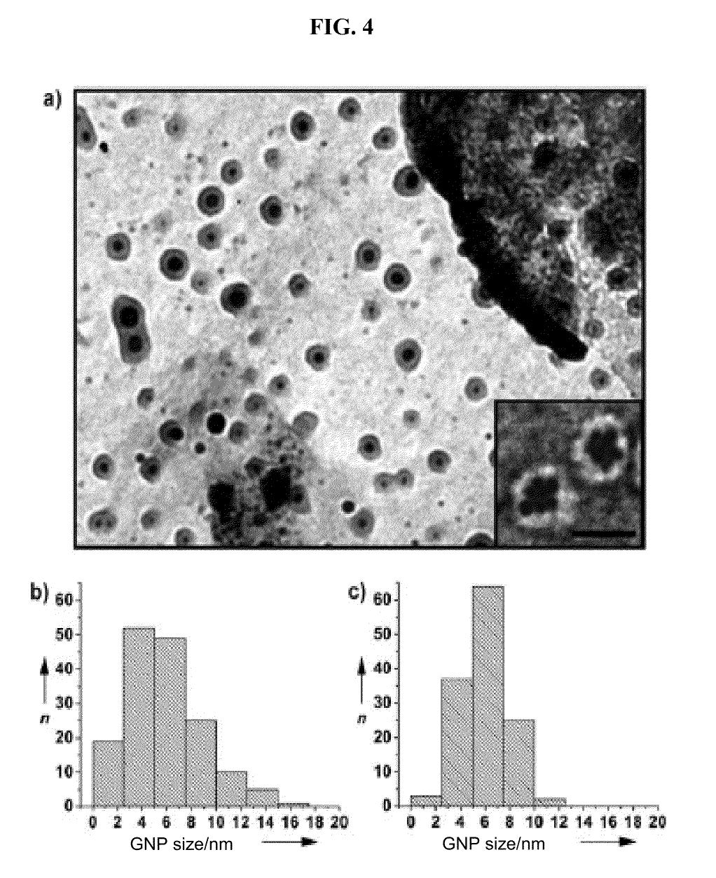

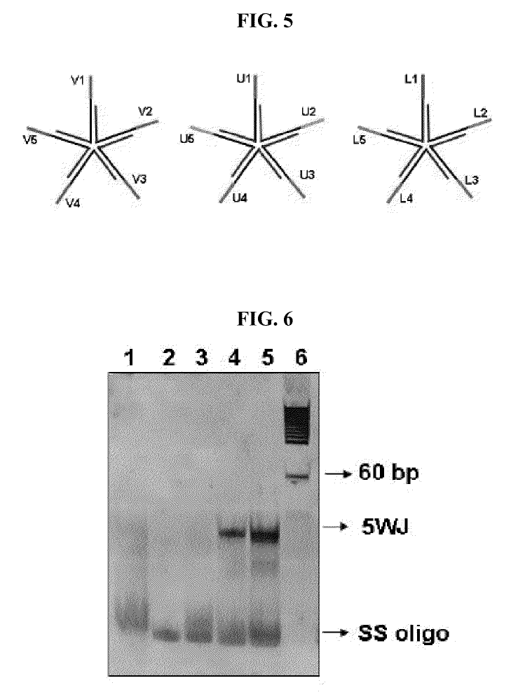

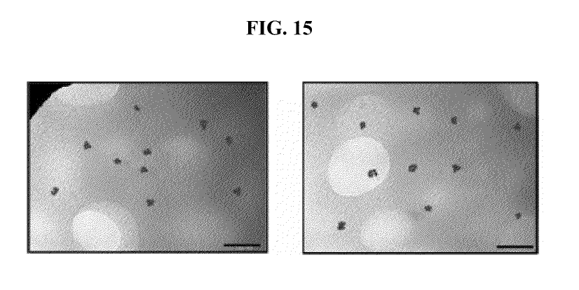

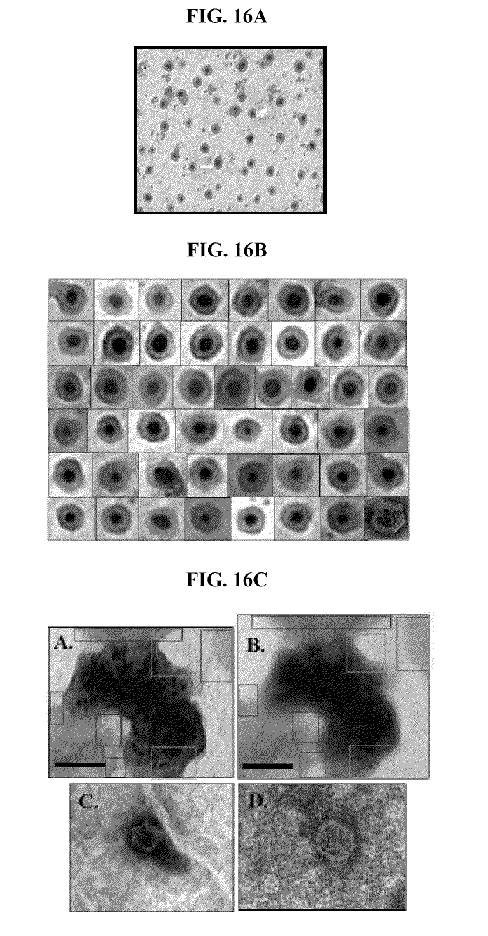

Encapsulation Studies. Citrate capped gold nanoparticles of diameters 1.5, 3.5 and 8 nm size were prepared using standard literature methods and used for encapsulation studies. 0.33 μM VU₅ and VL₅ were mixed in a 1:1 ratio in the presence of a solution of gold nanoparticles, annealed and ligated. The [2:5:5] complexes were separated from the free gold nanoparticles via size exclusion chromatography on a G-75 sephadex column and dialysis, and the resulting solution used for microscopy. 10 μL of the sample was added onto silicon monoxide TypeA, formvar grid and directly viewed under a TEM (FIG. 15). 1% aqueous uranyl acetate was used to negatively stain sample loaded air-dried grids and visualize the caged gold nanoclusters. (FIG. 4A and FIG. 16).

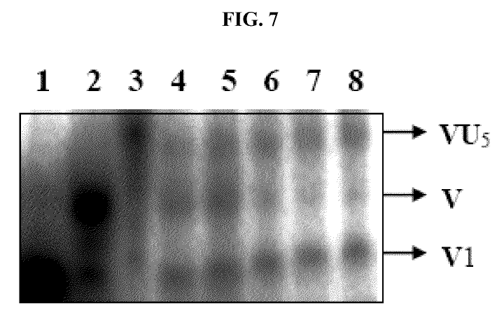

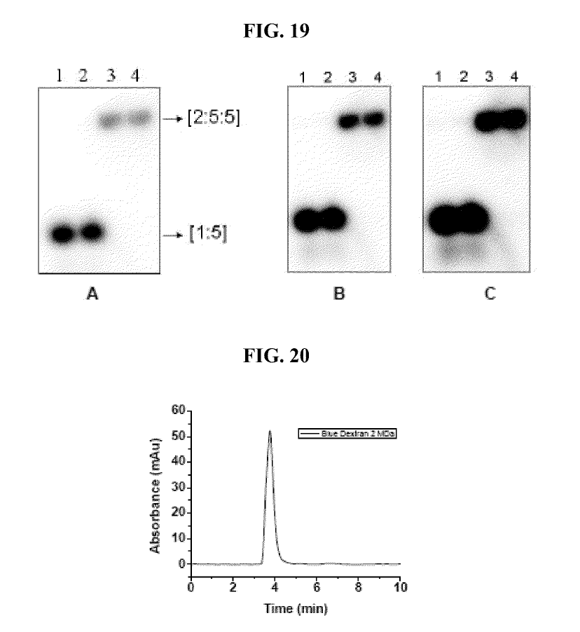

Gel electrophoresis. Native polyacrylamide gels containing 10 or 15% acrylamide [19:1 acrylamide/bisacrylamide] were used to study the mobilities of the 5WJs and [1:5] complexes. The running buffer consists of 100 mM Tris.HCI, pH 8.3, 89 mM boric acid, and 2 mM EDTA (TBE). Gels were run at 10V/cm and post run the gel was stained with Ethidium Bromide (1 μg/ml) and observed under a UV-illuminator (Alpha Imager, Alpha Innotech). 15% PAGE was used to show the formation of 5WJs (FIG. 2A & FIG. 6) and 10% PAGE to show [1:5] complexes (VU₅ and VL₅) (FIG. 2B). The [2:5:5] complex was characterized on 0.8% Agarose gel (FIG. 3A). For electrophoretic mobility shift assays, single stranded, V1 oligo was labeled with [γ-³²P] ATP and T4 Polynucleotide kinase. The labeled V1 was mixed with unlabeled V2-V5 in equimolar concentrations to make the V 5WJ. The labeled V 5WJ was mixed with unlabeled U 5WJ in molar ratios of 1:1, 1:2, 1:3, 1:4 and 1:5. Samples were electrophoresed through 10% native polyacrylamide gels in TBE (pH 8.3) at 10V/cm. The gel was dried and exposed to Fuji Phosphorlmager screen and the bands were visualized using the associated manufacturer software (FIG. 2C and FIG. 7). To show the formation of [2:5:5] complex in the indicated stoichiometry, ligated VU₅ and ligated VL₅ were complexed in molar ratios of 0.2:1, 0.5:1, 0.7:1 and 1:1. Samples were electrophoresed on 0.8% Agarose and the gel was stained with Ethidium Bromide (FIG. 3B). To calculate the yield, the [2:5:5] complex, I was synthesized from two complementary [1:5] complexes VU₅ and VL₅ which were assembled from radiolabeled V 5WJ. Samples were electrophoresed on 0.8% Agarose and exposed to Phosphorimager. (FIG. 19). The assembling yield of the final structure was calculated using the band intensity in gel using ImageJ software (NIH). (See Example 7). Yields are an average of at least 4 independent radiolabeled experiments.

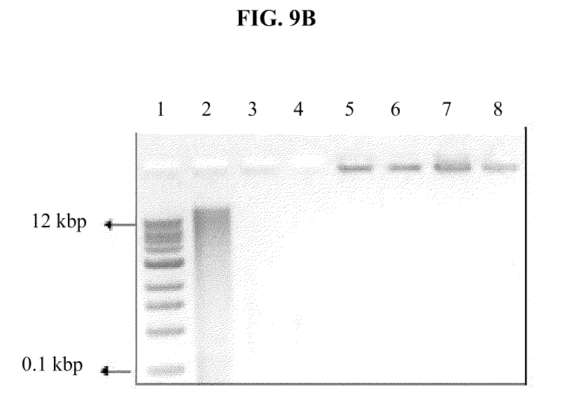

Exonuclease assay. All the intermediates, i.e., the single vertex, six vertex and twelve vertex species were subjected to exonuclease digestion (Lambda exonuclease, 3U, NEB) at 37° C. for 4 h and (T7 Exonuclease, 5U, NEB) at 25° C. for 3 h. The enzyme was removed by chloroform extraction and the samples were checked by gel electrophoresis. Same reactions were performed on genomic DNA isolated from HeLa cells as a control (FIG. 3C & SI FIG. 9).

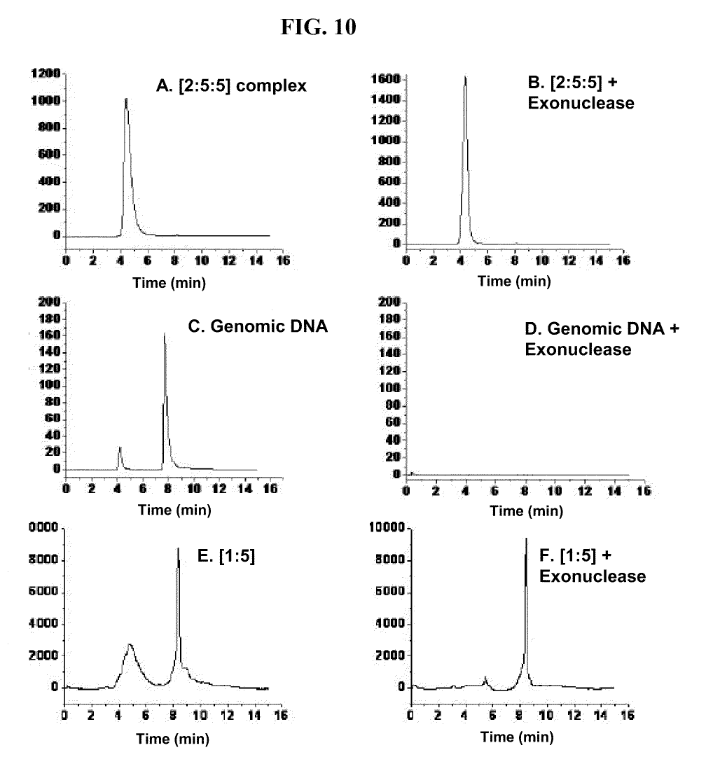

Size exclusion chromatography. The efficiency of the process was checked by HPLC on a size exclusion column (SEC-IIPLC). 4 nmoles (or 800 nM base molarity) of the relevant DNA sample were loaded on a BioSep-SEC 3000 IIPLC column and eluted isocratically with a flow rate of 0.8 mL/min (FIG. 10).

### Example 1

### Modular Assembly of DNA Polyhedra

We constructed the most complex DNA-based platonic solid, namely, an icosahedron, through a unique modular assembly strategy and demonstrated this functional aspect for DNA polyhedra by encapsulating gold nanoparticles (GNPs) from solution.

This modular assembly strategy to access complex polyhedra involves a stepwise amalgamation of discrete modules obtained from degenerate components. DNA icosahedra may be constructed from three distinct five-way-junction (5WJ) components V, U, and L, with programmable overhangs (FIG. 1A and Table 1). In FIG. 1A, the 5WJs V, U, and L are shown. The heavy black lines represent double-stranded regions, and the complementary overhangs are color-coded. In FIG. 1B, each half is formed from a central vertex 5WJ), V, and five equivalents of the 5WJ U or L. In FIG. 1C, the complex structure, I, is formed by the addition of an upper (VU₅) to a lower half (VL₅) in a 1:1 ratio.

Each 5WJ module, V, U, and L, is constructed from equimolar amounts of the respective five phosphorylated single strands (FIG. 2A and FIG. 6). At 20 μM, V was shown to form a complex with L in a 1:5 ratio (FIGS. 2A and 2B). FIG. 2A is a PAGE (10%) showing the formation of the 5WJ V and the formation of the [1:5] complexes VU₅ and VL₅ from 5WJs. Lane 1: DNA marker; lane 2: V1 oligonucleotide; lane 3: 5WJ V; lane 4: VU₅; lane 5: VL5. FIG. 2B presents gel electrophoresis showing the formation of the [1:5] complex VL₅ in the indicated stoichiometry. The radiolabeled 5WJ V was complexed with the unlabeled 5WJ L at different ratios. Samples were then subjected to electrophoresis on 10% native PAGE in TBE buffer and visualized with PhosphorImager. Lane 1: V+L; lane 2: V+2L; lane 3: V+3L; lane 4: V+4L; lane 5: V+5L; lane 6: 5WJ V (³²P-labeled V1 oligonucleotide).

The complementary module VU₅ was synthesized similarly from components V and U (see FIG. 7). At this stage, contiguously hybridized strands in VU₅ and VL₅ were ligated chemically with N-cyanoimidazole (NCI) to enhance stability.

When 5WJs of U attached to 3.5 nm gold nanoparticles were complexed with V in a 1:5 ratio and investigated by electron microscopy, several pentagonal arrangements of gold nanoparticles were observed in the [1:5] complex VU₅ (FIGS. 2C, 2D, 7 and 8). FIG. 2C is a representative transmission electron micrograph (TEM) of a gold-nanoparticlelabeled U 5WJ in a VU₅ complex. Scale bar: 20 nm. FIG. 2D is a defocused image of the same field; gold nanoparticles appear as white spheres as a result of defocusing. Scale bar: 20 nm. The average center-to-center distance between two gold nanoparticles that mark adjacent vertices (“a”) and nonadjacent vertices (“b”; see FIG. 7) in these pentagonal arrangements of VU₅ were a=8.8±1 nm (n=36) and b=13.7±1.7 (n=12). This result is consistent with the theoretical distances (a=8.3 nm, b=13.4 nm) in the proposed half-icosahedral, compacted, cup-shaped arrangements resulting from recognition between complementary overhangs U₅; and U₂, of adjacent U 5WJs in the complex VU₅.

The two different modular assemblies, VU₅ and VL₅, with ten identical overhangs each (the overhangs are complementary in the two assemblies), were shown to complex with each other in a 1:1 ratio. The contiguous termini were ligated again with NCI to yield a complex I with a 2:5:5 V/U/L stoichiometry in high yield (ca. 90%; FIGS. 3A, 3B). FIG. 3A is an agarose gel (0.8%) showing the formation of the [2:5:5] complex I from VU₅ and VL₅. Lane 1: ligated VU₅; lane 2: ligated VL₅; lane 3: the ligated [2:5:5] complex I. FIG. 3B is an agarose gel (0.8%) showing the formation of the [2:5:5] complex in the indicated stoichiometry from VU₅ and VL₅. Lane 1: ligated VL5; lane 2: the ligated [2:5:5] complex I; lane 3: VU₅/VL₅ (0.2:1); lane 4: VU₅/VL₅ (0.5:1); lane 5: VU₅/VL₅ (07:1); lane 6: VU₅/VL₅ (1:1).

The [2:5:5] complex I was resistant to both T7 and lambda exonucleases, which indicates the presence of circularized component DNA strands and is consistent with the highly catenated nature of the putative tertiary structure adopted by the complex (FIG. 3c; see also FIG. 9). FIG. 3c shows exonuclease treatment of the ligated [2:5:5] complex on 0.8% agarose gel. Lane 1: 0.5-12 kb DNA marker; lane 2: genomic DNA from HeLa cells not treated with the exonuclease; lane 3: genomic DNA treated with 3 U of lambda exonuclease for 4 h at 37° C.; lane 4: genomic DNA treated with 5 U of T7 exonuclease for 3 h at 25° C.; lane 5: the ligated [2:5:5] complex, not treated with lambda exonuclease; lane 6: the ligated [2:5:5] complex treated with 3 U of lambda exonuclease for 4 h at 37° C.; lane 7: the ligated [2:5:5] complex, not treated with T7 exonuclease; lane 8: the ligated [2:5:5] complex treated with 5 U of T7 exonuclease for 3 h at 25° C. Particle-sizing measurements on the [2:5:5] complex I by tapping-mode AFM gave a mean height distribution of (16.7±2.7) nm (n=130; FIG. 11). To obtain information on particle morphology, we adsorbed an aliquot of the specimen onto a carbon-coated, glow-discharged formvar support on a 400 mesh copper grid. The specimen was subjected subsequently to either platinum shadowing or negative staining with 1% uranyl acetate, and imaged by bright-field TEM under variable accelerating voltages (100/120/200 kV) by using a low-beam current. In both cases, several regular hexagonal and pentagonal features were observed, with C₃, and C₅ symmetries, respectively (FIG. 3D). Platinum-shadowed samples showing hexagonal (top left) and pentagonal (bottom left) features corresponding to C₃ and C₅ symmetries. (FIG. 3D) Scale bar: 20 nm. Shown on the right are the calculated theoretical dimensions (nm).

The observed cevian length of the pentagonal features was found to be (15.4±0.8) nm (n=135), and the observed tip-to-tip and side-to-side distances of the hexagonal features were (19.1±0.6) nm (n=131) and (17.3±0.4) nm (n=128), respectively. These dimensions were in excellent correlation with an icosahedral arrangement of the component helices in the [2:5:5] complex constructed with the software Matlab 7 (FIG. 13), for which DNA helices were assumed to be cylinders of diameter 2.3 nm. Importantly, the large numbers of hexagonal and pentagonal features indicate that the [2:5:5] complex could adopt an icosahedral tertiary structure of degree k=1, as observed for several viral particles.

To see whether the [2:5:5] complex was capable of functioning as capsules for other nanoscale entities, we mixed VU₅ and VL₅ in a 1:1 ratio in the presence of citrate-capped gold nanoparticles in a buffer. Following ligation with NCI, the free gold nanoparticles were separated from the [2:5:5] complex by size-exclusion chromatography on a G-75 sephadex column and dialysis. Visualization by TEM of an aliquot of the dialyzed solution of the [2:5:5] complex without staining showed that gold nanoparticles were present only in clusters of at least 6±2 particles (FIG. 15). Negative staining of the same samples with 1% aqueous uranyl acetate revealed the presence of several hexagonal and pentagonal [2:5:5] particles with a highly electron dense core under low magnification (50 kX; FIG. 4A; see also FIG. 16). At higher magnification, gold nanoclusters could be visualized clearly inside [2:5:5] cages of average sire (23±2) nm (FIG. 4A, inset). Furthermore, histograms of gold-nanoparticle sizes before and after encapsulation reveal a depletion of nanoparticles below 2.5 nm in diameter (FIGS. 4B, 4C). This result is consistent with an in-circle diameter of 2.8 nm of the triangular faces, as predicted for an icosahedral arrangement of helices in the [2:5:5] complex, and further indicates the overall effective porosity in these capsules. The minimum effective encapsulation volume per [2:5:5] complex is approximately 580 nm³ or approximately 53% of the computed void volume. Modules VU₅ or VL₅ alone were incapable of encapsulating gold nanoparticles (see the Supporting Information). However, encapsulation by a defective [1:5:5] complex created through a different assembly strategy (FIG. 17) and missing a single vertex gave an effective pore size of 3.5 nm. Thus, this assay for pore size in the bulk complex is sensitive to an overall loss of a single vertex within the structure.

### Example 2

### Stoichiometry of the [1:5] Complex VU₅ by Electron Microscopy

To address the issue of stoichiometry, we showed that the half icosahedron requires a stoichiometry of 1:5 V:U to form. We labeled one of the single stranded components (V1) with [γ-³²P] ATP. The corresponding 5WJ (V) comprising labeled V1 is shown in Lane 2 (FIG. 7). Labeled V was mixed with unlabeled U in increasing ratios 1:1, 1:2, 1:3, 1:4, 1:5 (FIG. 7, Lanes 4-8). Electrophoresis of the samples clearly showed that V complexes with U to form VU₅ at a ratio of 1:5. This is also indicative of the fact that this structure (VU₅) forms in a cooperative manner, i.e., addition of suboptimal amounts of U does not result in structures such as VUₙ(where n<5), but (VU₅+excess V).

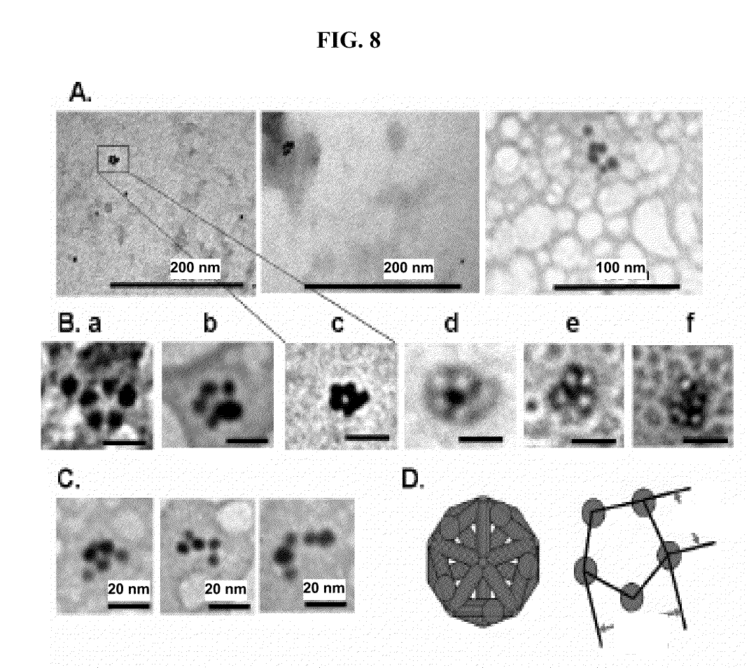

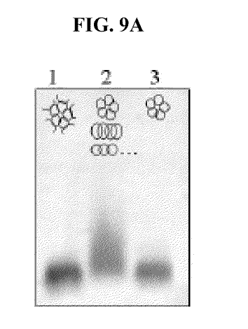

The gold nanoparticles are of average size of 3.5 nm. The average centre to centre distance between two gold nanoparticles marking adjacent vertices (‘a’) and non-adjacent vertices (‘b’) in the pentagonal arrangement in VU₅ are a=8.8±1 nm (n=36) and b=13.7±1.7 (n=12). FIG. 8A is a series of low magnification images showing the 1:5 complexes as a ring of 5 gold nanoparticles and the distribution of stray gold nanoparticles remaining post-dialysis. FIG. 8B is a series of high magnification images of gold nanoparticle labeled [1:5] complexes: (a-c) are focused images showing the highly electron dense gold nanoparticles. (d-f) are defocused images so that the centre of individual gold nanoparticles (that appear as white spheres due to defocusing) may be clearly visualized. (c & d) are focused and defocused images respectively of the same field to illustrate this point. Scale Bar: 20 run. FIG. 8C is a series of high magnification images of [1:5] complexes showing partially folded intermediates; however these occur with much less frequency. FIG. 8D is a schematic showing the apparent orientation of the gold nanoparticles on the relevant helices of the predicted pre-folded [1:5] complex consistent with the experimentally obtained distances for ‘a’ and ‘b’.

We observed that all the [1:5] species are not entirely topologically folded. We could observe rare occurrences of [1:5] species where one gold nanoparticle does not occupy the expected fifth vertex of the pentagon, but hangs onto one of the terminal 5WJs forming a group of five particles. The presence of such intermediates suggests that the [1:5] complex probably undergoes an internal folding event via recognition of complementary sites on different U 5WJs. This is consistent with our findings that longer equilibration times at the formation-step of the [1:5] complex results in better yields of the [2:5:5] complex. It is possible that upon further complexation to give the [2:5:5] species, probably due to further equilibration at the subsequent step and/or the high probability of a preorganized complementary site being present on the partner [1:5] species, such partially folded forms undergo complete hybridization. The results of this experiment validate that the [1:5] complex is pre-folded into a cup-shaped, half icosahedral arrangement of its component helices.

### Example 3

### Exonuclease Digestion of [1:5] and [2:5:5] Complexes

To show that the [1:5] complex is made up of catenated (circularized) oligonucleotides following its ligation, we subjected this complex to treatment with Lambda exonuclease and T7 exonuclease. If there are any free unligated termini, Lambda exonuclease would degrade the double-stranded portion into corresponding nucleotides. Further it is also active on ss DNA, however this is less efficient. If there are any nicks during the hybridization of the complex, T7 exonuclease would degrade the strands into the corresponding nucleotides. The results are shown in FIG. 9A (Lane 1. ligated [1:5] without treatment with exonuclease; Lane 2: ligated [1:5] treated with 5U of T7 exonuclease for 3 h at 25° C.; lane 3: ligated [1:5] complex treated with 3 U of Lambda exonuclease for 4 h at 37° C.) Importantly, we observe (i) a single well-defined product obtained from treatment with Lambda exonuclease corresponding to the pentacatenated species and (ii) the smear obtained from subjecting the [1:5] to T7 exonuclease, showing that multiple products arise from a single scaffold due to nicks (FIG. 9A). These could be bicatenated, tricatenated, tetracatenated in addition to pentacatenated species. This further strengthens the topology and stoichiometric purity of the [1:5] complex.

The buffer conditions for Lambda (New England Biolabs) and T7 exonucleases (New England Biolabs) are not compatible to allow simultaneous use without compromising the efficiency of one of them. We have therefore subjected the [2:5:5] complex to digestion with Lambda exonuclease and T7 exonuclease sequentially (FIG. 9B). Lanes 2-4 show genomic DNA isolated from HeLa cells as a positive control indicating completeness of digestion of duplex fragments using the indicated protocols for both Lambda and T7 exonucleases. Treatment with Lambda exonuclease resulted in insignificant degradation of the [2:5:5] complex from a comparison of the intensities of Lambda exonuclease untreated product with respect to the Lambda treated product, not accounting for sample loss during precipitation. In addition, treatment with T7 exonuclease revealed that a considerable amount (92%) of the [2:5:5] complex from a comparison of the intensities of T7 treated product with respect to the Lambda treated product, not accounting for sample loss during precipitation, indicating a large percentage of successful circularizations.

These experiments were further confirmed by analysis of the same mixtures by size exclusion chromatography (SEC-HPLC). FIG. 10 shows that the ligated [2:5:5] complex shows a single, symmetric peak with a retention time of 4.3 min, characteristic of a single species. Importantly, the pore size of the column is 30 nm and this species is not observed in the void volume (Vₒ=1.9 ml [as specified by the manufacturer]). Taken together, this indicates this complex is not a high molecular weight, nonspecific aggregate. As a control, genomic DNA, a high molecular weight polymer, shows at least two bands, both of which are not symmetric, with retention times of 4.2 and 7.8 min (See FIG. 10, panel C). When the [2:5:5] complex is subjected to exonuclease treatment, the SEC-HPLC profile showed no change (FIG. 10, panel B). However, the SEC-HPLC profile of genomic DNA after treatment with exonuclease (FIG. 10, panel D) using the same protocol showed a complete degradation and only residual small fragments are observed.

### Example 4

### Electron Microscopy

Crowded field images of the [2:5:5] complex were taken using three different electron microscopes operating at three different voltages replicated on several different samples. FIG. 12A is an image taken on Tecnai F20 FEI machine, operating voltage 200 kV, magnification-100 kX, Scale bar: 100 nm. FIG. 12B is an image taken on JEOL 100 CX II machine, operating voltage 80 kV, magnification 50 k X, Scale bar: 100 nm. FIG. 12C is an image taken on Tecnai 12 Biotwin, operating voltage 100 kV, magnification 135 kX, Scale bar: 100 nm. In this figure, the image has been intentionally defocused to obtain the edges of the particles formed due to Fresnel diffraction which indicates that this shape is due to the underlying structured particle and not an artifact of uranyl acetate staining inhomogeneity. These crowded field images indicated that the [2:5:5] complex showed well-formed particles of the predicted dimensions, showing C3 and C5 axes of symmetry, indicating the reproducibility and propensity for the structure to form well.

### Example 5

### Matlab Model for the Construction and Characterization of DNA Icosahedron

Matlab was used to construct a model of the DNA icosahedron. The main aim of constructing this model is to calculate distances and dimensions of the DNA icosahedron. DNA double helices were assumed to be cylinders of 2.3 nm diameter and 8.8 nm in length (26 nucleotides). Using Matlab 7, we modeled the icosahedron using cylinders of 2.3 nm diameter and uniform length in two different ways.

From a regular icosahedron, we consider the basic unit of the structure to be an equilateral triangle, ABC, constructed from three cylinders aa′, bb′, and cc′ of length 1. FIG. 13(I) shows the basic triangular unit constructed using the overlapping cylinder model Icosahedron Modeling in Matlab 7. FIG. 13(II) is a view of the resulting icosahedron showing the C-3 axis of symmetry. FIG. 13(III) is a view of the resulting icosahedron showing the C-5 axis of symmetry and cevian length, w. From FIG. 13, it is apparent that the effective length, l_{eff}, of the side of the triangle ABC determines the size of the resultant icosahedron where aa′=bb′=cc′=8.8 nm. The Icosahedron can therefore be modeled in two simplistic ways from a cylinder of length 8.8 nm and diameter 2.3 nm.

In the first model, FIG. 13, the cylinders are fitted over regular icosahedral coordinates assuming no overlap between them at the vertices. In this model, l_{eff} of each triangular side (AB, BC or AC) is lᵣ+2d (where d=Aa, Ba′, Bb, Cb′, Cc or Ac′). Therefore, in a model that assumes non-overlapping cylinders, the effective edge length in the icosahedron is larger than the length of the cylinder lᵣ.

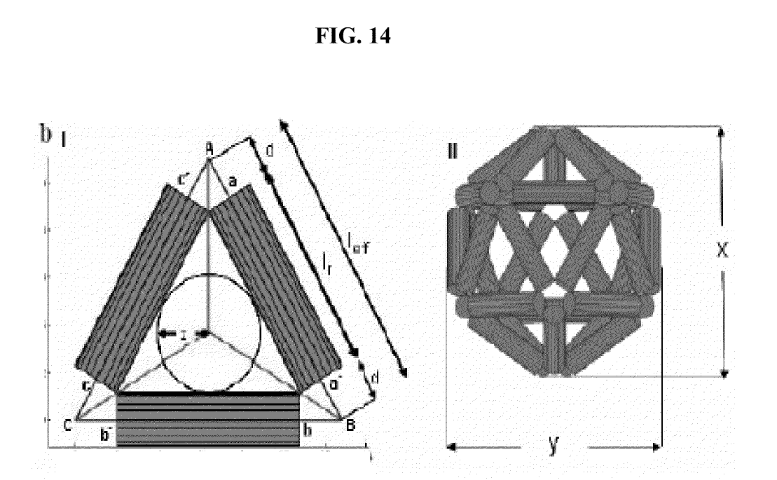

In the second model (FIG. 14), the basic triangular unit constructed from these cylinders does not allow for any additional length, and assumes that the dimensions of the triangular sides in the icosahedron, l_{eff}, is equal to the length of the cylinder, lᵣ. This necessitates a 50% overlap of the cylinders at their vertices. The dimensions for such a model are,

(i) The Tip to tip distance, x=18.8 nm

(ii) Side to side distance, y=16.5 nm

(iii) In-circle radius, z=1.4 nm

iv) Cevian length of the pentagon, w=15.6 nm

(v) Effective encapsulatable volume 1088.6 nm³.

The latter model is more reasonable, because the cylinders are in reality helices with major and minor grooves. Thus, at the vertices, an “overlap” is possible where part of the backbone of one of the helices can be accommodated in the major groove of the adjacent helix as also seen in a model of a DNA tetrahedron.

This model dictates an upper limit of 50% overlap between the cylinders. This is also consistent with the B-DNA double helix, where the upper limit to “overlap” is 1.2 nm (50%). The maximal length along the cylinders involved in the overlap, implying accommodation in the major groove, is 2.6 nm, consistent with 7 base-pairs of B-DNA. This is in reasonable correspondence with an overlap of 6 base pairs as seen in the vertices of a model of the DNA tetrahedron.

### Example 6

### Encapsulation of Gold Nanoparticles by the [2:5:5] Complex

FIG. 15 shows bright-field TEM at 80 kV of [2:5:5] complex containing fraction V from size exclusion column performed post-encapsulation showing clusters of GNP (without staining) TEM images show that gold nanoparticle clusters are present inside uranyl acetate repellent cages roughly 20-25 nm size. DNA is well known to repel uranyl acetate and stain negatively. Thus the cluster of gold nanoparticles are indeed present in DNA cages formed by the [2:5:5] complexes.

Crowded field images of gold nanoparticles encapsulated inside [2:5:5] complexes containing metallic centers are shown in FIG. 16. TEM images of [2:5:5] complexes post-encapsulation, stained with uranyl acetate indicating that every [2:5:5] complex encloses a highly electron dense core (i.e., gold nanoparticles present inside hexagonal and pentagonal species in a crowded field). As this is a crowded field, it is in low resolution and therefore the gold nanoparticle cluster appears as a highly electron dense core inside negatively stained pentagonal and hexagonal DNA shells of the predicted dimensions.

From our experiments, the percentage of gold nanoparticles remaining unincorporated has a lower estimate of 19% provided every icosahedron shows encapsulation behavior. This is a reasonable assumption given the crowded field images (FIG. 16A and FIG. 4 show typical micrographs) of GNP-containing icosahedra, where the percentage of DNA particles without a metallic core is <1% (n=123). The stoichiometry of [1:5] complexes: GNPs prior to encapsulation is estimated at 1:3.4.

We have provided representative zoomed images of the particles shown in FIG. 16B, but as the data is taken at low resolution, the individual gold nanoparticles are not visible. As well, many of the hexagonal and pentagonal vertices of the DNA cages are blurred. However under high resolution TEM, (FIG. 16B, bottom right hand corner image) the individual gold nanoparticles in a given cluster can be observed.

In the high-resolution picture of the same sample (FIG. 16B), pentagonal features of the DNA cage encapsulating individual gold nanoparticles is clearly observable—but due to the high magnification we cannot show a crowded field.

To confirm whether indeed the gold nanoparticles were inside the icosahedral DNA complexes, or whether the uranyl acetate simply formed a halo (an artifact of negative stain methodology) around a pre-clustered GNP aggregate, we used a Tecnai 12 BioTwin, FEI Co, Netherlands, TEM equipped with a twin lens configuration which enables the defocusing of one of the lenses. If there is a periodic structure or an architecture or a capsid, we will observe an interference pattern due to Fresnel diffraction on the boundaries of the structure in question. This will result in these boundaries showing double lines (due to diffraction). In the case of an artifact due to a halo, that border will show no Fresnel diffraction. We present two independent experiments and their respective defocused images. The regions of interest are marked in squares that show Fresnel diffraction on the stained region surrounding the GNPs. The artifact halos do not show any Fresnel diffraction pattern (FIG. 16C).

Encapsulation Studies on the [1:5] species. The [1:5] complexes were mixed with the gold nanoparticles and the resultant solution after equilibration was dialyzed and the dialyzed fractions were observed in EM at a 100-fold greater concentration than the [2:5:5] samples. It was observed that after dialysis, even at these concentrations, negligible gold nanoparticles were observed and that the rare gold nanoparticle that was observed was always solitary (data not shown). This indicates that the individual [1:5] complexes are not capable of encapsulating the species from the solution. The observation of rare occurrence of gold nanoparticles is because dialysis is an equilibrium method, and there will always be some nanoparticles remaining.

50 μL of 0.33 μM solution of [1:5] (VU₅/VL₅) was mixed with 200 μL of a solution containing mixture of (1.5, 3.5 and 8 nm size) gold nanoparticles. The resultant mixture was annealed at 45° C. for 4 h, and then cooled at the rate of 5° C./15 min to room temperature. The resultant mixture was allowed to equilibrate at 4° C. for 2 days and the resulting mixture was dialyzed against 100 mM NaCl, 1 mM MgCl₂ for 10 h at room temperature. The aliquot from the dialyzed solution was analyzed by TEM using the same procedure as mentioned for [2:5:5] complex in the experimental section.

Encapsulation Studies on [1:5:5] complex. Due to the cooperativity associated with the self-assembly principle in the DNA [2:5:5] complex, we could not make defective icosahedra, by mixing VU₄ (as we obtain VU₅ and extra V) and VL₅. So instead, we made [1:5:5] complexes by incubating VU₅ with 5 equivalents of L and missing out vertex V.

20 μL of VU₅ (4 μM) and 20 μl of L 5WJ (20 μM) were mixed in sodium phosphate buffer (10 mM), 1 mM MgC₁₂ and 100 mM NaCl. The resultant solution was heated to 45° C. for 4 hours and then annealed at the rate of 0.33° C./min up to 20° C. The sample was allowed to equilibrate at 4° C. for 2 days after which it was ligated using NCI. The similar experiment was carried out in presence of gold nanoparticles of sizes (1.5, 3.5 and 8 nm size). After ligation the resulting solution was dialyzed and the aliquot of this dialyzed sample dried on an EM carbon coated copper grid, stained with 1% uranyl acetate for 2 sec and viewed under bright field TEM (JEOL 100 CX II, 80 kV and Tecnai G12 Biotwin, 100 W). The sizes of the gold nanoparticles present in the clusters were measured and plotted for comparison (FIG. 17).

Encapsulation of gold nanoparticles was performed by mixing VU₅ with 5L in an environment of gold nanoparticles. We observe that in such defective [1:5:5] complexes, gold nanoparticles show depletion in larger particles indicating a tangibly increased porosity. Importantly, several observations of particles dissociating from a cluster was observed unlike in the intact [2:5:5] complexes where particles tend to be confined to a cluster (FIG. 17).

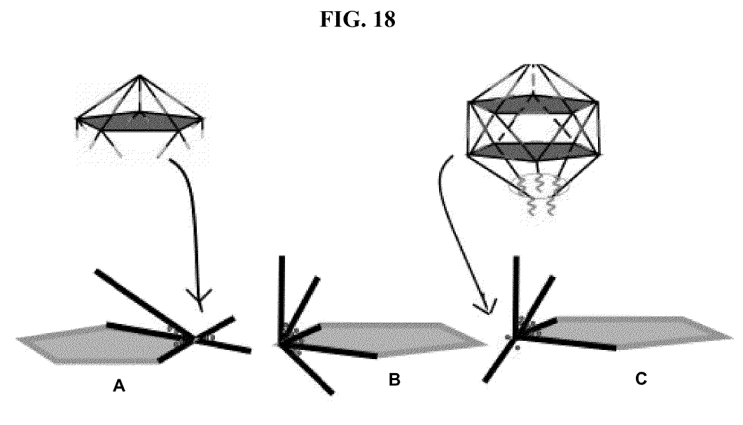

We present a possible explanation for the counter-intuitive, weakly encapsulatable property of the [1:5:5] complex given the complete absence of encapsulation property by the [1:5] complex. There could be two possible reasons, (i) the [1:5:5] complex could have a larger encapsulable volume than the [1:5] complex and/or (ii) the effective pore size opposite vertex V is somehow smaller in the [1:5:5] complex than the [1:5] complex. We provide a possible molecular explanation for the latter (FIG. 18). In the 5WJs of U in the [1:5] complex in a half icosahedral shape, 3 arms are bent inwards to form the half icosahedral structure and 2 arms are free. It is highly likely that the two free arms could be splayed outwards (driven by electrostatic repulsion) leaving the lower cavity nicely open. In the [1:5:5] complex, the L 5WJs that flank the large cavity have 4 out of 5 arms pre-organized and bent in one direction. It has been shown that coordination of multiple M²⁺ metal ions in binding pockets created in between spatially orientated arm junctions are crucial to stability and folding of many arm junctions in RNA molecules and DNA assemblies. It is likely that the organization of four arms of the L 5WJ on the [1:5:5] complex causes some pre-organization of the fifth arm to bend inwards to create a binding pocket for Mg²⁺ ions to achieve the required pre-organization for 4 arms to bend in the same direction. Such inward bending of the free arms of L 5WJs in the [1:5:5] complex could decrease the effective pore size compared to the [1:5] complex. Inward bending could also be stabilized by non-specific interactions between the 5′ overhangs of the bent arms in the L 5WJs.

### Example 7

### Efficiency and Yield of [2:5:5] Complex

Gels of the reaction mixture of the [2:5:5] complex showed a band of highly retarded mobility (FIGS. 3A and 3B). This band on SEC-HPLC showed only a single sharp peak which was distinct from the [1:5] components (FIG. 10) thus ruling out the possibility that the low mobility band could be a mixture of high molecular weight polymers of [1:5] complexes. Analysis by EtBr staining of gels as well as SEC-HPLC of the VU₅+VL₅ reaction mixture showed that they complexed with each other in a fixed stoichiometry and there were no detectable starting materials left over (VU₅, 5WJs or ss oligonucleotide component DNA) (FIG. 19).

Yield of this reaction was measured by radiolabeling V1 to give radiolabeled V 5WJ. VU₅ and VL₅ were formed from radiolabeled V 5WJ and subsequently complexed to give [2:5:5] complex that was then quantified by pixel counting including the background in the entire lane. Samples were electrophoresed on 0.8% Agarose in TAE buffer and visualized using PhosphorImager. Lane 1. ligated radiolabeled VU₅; lane 2. ligated radiolabeled VL₅; lane 3. Ligated [2:5:5] formed by complexation of radiolabeled VU₅ and unlabeled VL₅; lane 4. Ligated [2:5:5] formed by complexation of radiolabeled VL₅ and unlabeled VU₅. (FIGS. 19B, 19C).

The results show a yield of 90% by radiolabeling. However, we must emphasize that no other distinct band/peak was seen other than the [2:5:5] complex by gel electrophoresis (EtBr staining and radiolabeling) and SEC.

### Example 8

### Absolute Stoichiometry from Size Exclusion Chromatography

We have performed SEC-HPLC on the ligated reaction mixture as is, in order to see if the low mobility band has an absolute or relative stoichiometry of 2:5:5 V:U:L. The SEC column that we have used is the BioSep-SEC-S3000 (Phenomenex, Catalog no: OOH-2146-EO) with column dimensions 300 mm×4.6 mm and total column volume (Vc)=4.98 mL and Column material: Hydrophilic bonded Silica. The size of the packing beads is 5 μm and the pore size in these beads is 29 nm. (Journal of Chromatography A, 1992, 599:2533). As specified by the manufacturer, for a well-packed column, the void volume Vo is −30% Vc, i.e., 1.5 mL.

Measurement of Column Vo. Nevertheless, we have experimentally determined the void volume (Vo) of our specific column using a standard method of injecting Blue Dextran (molecular weight 2 MegaDalton) (FIG. 20). Being a very large polymer with R_{H} of 27 nm, and therefore a hydrodynamic size 54 nm, Blue Dextran only passes through the inter-bead spacing (the pore size of our SEC column is 29 nm) and elutes in the void volume of the column. At a flow rate of 0.5 mL/min, the Dextran Blue trace shows a retention time of 3.8 min which gives a Vo of 1.9 mL.

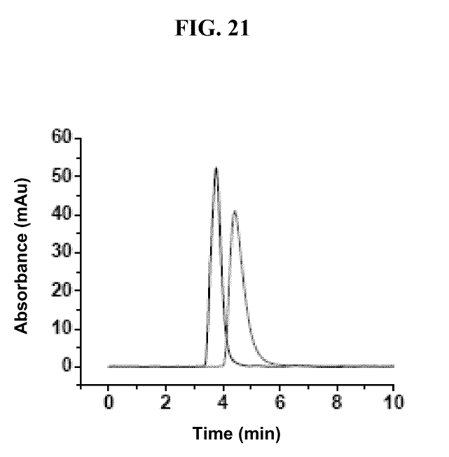

When we run the reaction product of the 1:1 mixture of VU₅ and VL₅ (both are 269 kDa each) on this column the [2:5:5] complex, I elutes with a retention time of 4.3 min which is distinctly beyond the void volume of the column (Overlaid traces, FIG. 21).

The column that we have used has an exclusion limit of 15 kDa-700 kDa. This indicates that the molecules within 15-700 kDa can be separated using this column and molecules in the range of 200-500 kDa will be resolved best. Molecules above 700 kDa will be eluted in the void volume. The retention time of the [2:5:5] complex indicates that since its elution volume is greater than Vo, that its molecular weight cannot be greater than 700 kDa. Importantly, the monomeric VU₅ or VL₅ show a sharp peak at 8 min (FIG. 10). That means the [2:5:5] complex is larger than a monomer (269 KDa). Its retention time is also consistent with this. A [4:10:10] complex (i.e., a tetramer) would have a molecular weight 1.076 MDa, which is well above 700 kDa and should elute in the void volume. A trimer of any combination of VU₅ and VL₅ will be 806 kDa and should also elute in the void volume.

However, a dimer of VU₅ and VL₅, would have a molecular weight of 538 kDa, fit with the obtained relative stoichiometry, will not elute in the void volume, will elute out before the [1:5] complex and would be expected to elute closer to the 700 kDa molecular weight peak (i.e., nearer the void volume). The SEC comparison of Blue dextran and [2:5:5] complex, I shows that I elutes at a retention time of 4.3 min and not in the retention time corresponding to the void volume proves that it is not even a trimer.

Importantly, the smallest aggregate with the established relative stoichiometry would be a [4:10:10] complex that would elute in the void volume—along with Blue dextran. These observations clearly show that the [2:5:5] (538 kDa) and [1:5] (269 kDa) complexes can be cleanly separated on the size exclusion column and their retention times are well within the optimal separation range and in accordance with the expected separation based on the SEC column characteristics. The observation of the single, symmetric peak for the [2:5:5] complex, not within the void volume of the column, confirms the presence of a single molecular weight species less than 700 KDa. Also, the fact that the [2:5:5] complex elutes after the void volume indicates that it is smaller than the pore size (29 nm) and larger than a [1:5] species (12×1 5×8 nm). This is consistent with the measured size of the [2:5:5] complex by TEM.

### REFERENCES

1. a) J. Chen, N. C. Seeman, Nature, 1991, 350:631-633; b) F. A. Aldaye, H. F. Sleiman, J. Am. Chem. Soc., 2007, 129:13376-13377.
2. a) R. P. Goodman, L A. T. Schaap, C. F. Tardin, C. M. Erben, M. Berry, C. E Schmidt, A. J. Turberfield, Science, 2005, 310:1661-1665; b) C. M. Erben, R. P. Goodman, A. J. Turberfield, Angew, Chem., 2006, 118:7574-7577; Angew, Chem. Int., Ed. 2006, 45:7414-7417; c) R. P. Goodman, M. Heilemann, S. Doose, C. M. Erben, A. N. Kapanidis, A. J. Turberfield, Nat. Nanotechnol., 2008, 3:93-96.
3. a) Y. He, T. He, M. Su, C. Zhang, A. E. Ribbe, W. Jiang, C. Mao, Nature, 2008, 452:198-202; b) J. Zimmermann, M. P. J. Cebulla, Monninghoff, G. V. Kiedrowski, Angew, Chem., 2008, 120:3682-3686; Angew, Chem. Int., Ed. 2008, 47, 3626-3630.
4. Y. Z. hang, N. C. Seeman, J. Ana. Chem. Soc., 1994, 116:1661-1669.
5. a) W. M. Shih, J. D. Ouispe, G. E Joyce, Nature, 2004, 427:618-621; b) E F. Andersen, B. Knudson, C. L. P. Oliveira, R. F. Frohlich, D. Kruger, J. Bungert, M. Agbandje-McKenna, R. Mckenna, S. Juul, C. Veigaard, J. Koch, J. L. Rubinstein, B. Guldbrandtsen, M. S. Hede, G. Karlsson, A. H. Andersen, J. S. Pedersen, B. R. Knudsen, Nucleic Acids Res., 2008, 36:1113-1119.
6. J. L. Kadrmas, A. J. Ravin, N. B. Leontis, Nucleic Acids Res., 1995, 23:2212-2222.
7. Y. Wang, J. E. Mueller, B. Kemper, N. C. Seeman, Biochemistry, 1991, 30:5667-5674.
8. K. J. Luebke, P. B. Dervan, Nucleic Acids Res., 1992, 20:3005-3009.
9. J. Qi, R. H. Shafer, Nucleic Acids Res., 2005, 33:3185-3192.
10. “Principles of Virus Structure”: S. C. Harrison in Field's Virology (Eds.: D. M. Knipe, P. M. Howley), 5th ed., Lippincott Williams & Wilkins, Philadelphia, 2007, 59-98.
11. D. D. Caspar, A. Klug, Cold Spring Harbor Symp. Quant. Biol., 1962, 27:1-24.
12. C. A. Mirkin, Inorg. Chem., 2000, 39:2258-2272.
13. D. G. Duff, A. Baiker, P P Edwards, J Chem. Soc. Chem. Commun., 1993, 96-98.
14. S. Schamm, C. Bonafos, H. Coffin, N. Cherkashin, M. Carrada, A. G. Ben, A. Clayerie, M. 'Ience, C. Colliex, Ultramicroscopy, 2008, 108:346-357.
15. C. Zhang, M. Su, Y. He, X. Zhao, P. Fang, A. E. Ribbe, W. Jiang, C. Mao, Proc. Natl. Acad. Sci. USA, 2008, 105:10665-10669.
16. H. B. Ghodke, R. Krishnan, K. Vignesh, G. V. P. Kumar, C. Narayana, Y. Krishnan, Angew, Chem., 2007, 119:2700-2703; Angew, Chem. Int. Ed. 2007, 46:2646-2649.

### EQUIVALENTS

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All references cited herein are incorporated by reference in their entireties and for all purposes to the same extent as if each individual publication, patent, or patent application was specifically and individually incorporated by reference in its entirety for all purposes.

Other embodiments are set forth in the following claims.

What is claimed is:

1. An iscosahedral nanocapsule comprising:
(a) one or more first five-way-junction (5WJ) modules each having a star-like configuration, wherein each first 5WJ module comprises five single-stranded oligonucleotides;
(b) one or more second 5WJ modules each having a star-like configuration, wherein each second 5WJ module comprises five single-stranded oligonucleotides; and
(c) one or more third 5WJ modules each having a star-like configuration, wherein each third 5WJ module comprises five single-stranded oligonucleotides;
wherein the first 5WJ modules, the second 5WJ modules, and the third 5WJ modules are configured to interact to form an icosahedral nanocapsule.

2. The nanocapsule of claim 1, wherein the five-single stranded oligonucleotides in each of the first, second, and third 5WJ modules are present in equimolar amounts.

3. The nanocapsule of claim 1 having two first 5WJ modules, five second 5WJ modules, and five third 5WJ modules.

4. The nanocapsule of claim 3, wherein each of the five single-stranded oligonucleotides in each of the first, second, and third 5WJ modules is configured to hybridize in an antiparallel orientation to another of the oligonucleotides over a portion of its length to form a partially double-stranded structure with five cohesive ends.

5. The nanocapsule of claim 4, wherein one of the cohesive ends of each of the second 5WJ modules is configured to specifically hybridize with the five cohesive ends of a first 5WJ module and two complementary cohesive ends of adjacent 5WJ modules are configured to specifically hybridize;
one of the cohesive ends of each of the third 5WJ modules is configured to specifically hybridize with the five cohesive ends of a first 5WJ module and two complementary cohesive ends of adjacent third 5WJ modules are configured to specifically hybridize; and
two cohesive ends in each second 5WJ module are configured to specifically hybridize with two complementary cohesive ends in each third 5WJ module to form the DNA nanocapsule.

6. The nanocapsule of claim 4, wherein the cohesive ends are from 4 to 16 nucleotides in length.

7. The nanocapsule of claim 1, wherein the oligonucleotides are ligated.

8. The nanocapsule of claim 1, wherein each of the oligonucleotides is from 10 to 250 nucleotides in length.

9. The nanocapsule of claim 1, wherein one or more of the oligonucleotides in one or more of the first, second or third 5WJ modules comprises one or more unpaired bases at the vertex of the star-like configuration.

10. The nanocapsule of claim 1, wherein the icosahedral nanocapsule has a tip-to-tip distance of 8 to 200 nm and a side-to-side distance of 8 to 200 nm.

11. The nanocapsule of claim 1, wherein the nanocapsule has a pore size from 0.5 to 10 nm.

12. The nanocapsule of claim 1, wherein each edge of the icosahedral nanocapsule has a length from about 8 to 150 nucleotides.

13. The nanocapsule of claim 1, wherein each edge of the icosahedral nanocapsule has a length from about 3.4 to 50 nm.

14. The nanocapsule of claim 1, wherein the icosahedron has an internal encapsulation volume of at least 400 nm³.

15. The nanocapsule of claim 1, further comprising one or more agents encapsulated in the nanocapsule.

16. A half-icosahedral structure for the modular assembly of icosahedral nanocapsules, the structure comprising:
(a) one first 5WJ module having a star-like configuration, wherein each first 5WJ module comprises five single-stranded oligonucleotides;
(b) five second 5WJ modules each having a star-like configuration, wherein each second 5WJ module comprises five single-stranded oligonucleotides;
wherein the first 5WJ module and the second 5WJ modules are configured to interact to form a half-icosahedral structure.

17. A method for assembling an icosahedral nanocapsule, the method comprising:
(a) combining one or more first 5WJ modules with one or more second 5WJ modules to form a first six vertex structure, wherein each 5WJ module has a star-like configuration and comprises five single-stranded oligonucleotides;
(b) combining one or more third 5WJ modules with one or more fourth 5WJ modules to form a second six vertex structure, wherein each 5WJ module has a star-like configuration and comprises five single-stranded oligonucleotides, and wherein the first 5WJ module and the third 5WJ module may be the same or different; and
(c) combining the first and second six vertex structures to form an icosahedral nanocapsule.

18. The method of claim 17, wherein each of the five single-stranded oligonucleotides in each of the 5WJ modules hybridizes in an antiparallel orientation to another of the oligonucleotides over a portion of its length to form a partially double-stranded structure with five cohesive ends.

19. The method of claim 18, wherein the icosahedral nanocapsule has one first 5WJ module, five second 5WJ modules, one third 5WJ module, and five fourth 5WJ modules.

20. The method of claim 19, wherein combining the first 5WJ module with five second 5WJ modules to form a first six vertex structure is by specific hybridization of one of the cohesive ends of each of the second 5WJ modules with the five cohesive ends of a first 5WJ module and specific hybridization of two complementary cohesive ends on adjacent second 5WJ modules;
combining the third 5WJ module with five fourth 5WJ modules to form a second six vertex structure is by specific hybridization of one of the cohesive ends of each of the second 5WJ modules with the five cohesive ends of a third 5WJ module and specific hybridization of two complementary cohesive ends on adjacent second 5WJ modules; and
combining the first and second six vertex structures is by specific hybridization of two cohesive ends in each second 5WJ module with two complementary cohesive ends in each fourth 5WJ module.


SEQUENCE LISTING (15 sequences)

SEQ ID NO: 1 - DNA, 26 bp, Artificial Sequence
  Description of Artificial Sequence Synthetic construct
gcctggtgccaccggtgacgttccgc

SEQ ID NO: 2 - DNA, 26 bp, Artificial Sequence
  Description of Artificial Sequence Synthetic construct
gcctggtgccccgcgtcctcaccggt

SEQ ID NO: 3 - DNA, 28 bp, Artificial Sequence
  Description of Artificial Sequence Synthetic construct
gcctggtgccgccacgctttggacgcgg

SEQ ID NO: 4 - DNA, 28 bp, Artificial Sequence
  Description of Artificial Sequence Synthetic construct
gcctggtgccgcgagtgcaaagcgtggc

SEQ ID NO: 5 - DNA, 28 bp, Artificial Sequence
  Description of Artificial Sequence Synthetic construct
gcctggtgccgcggaacgaagcactcgc

SEQ ID NO: 6 - DNA, 28 bp, Artificial Sequence
  Description of Artificial Sequence Synthetic construct
ggcaccaggcgcggaacgaagcactcgc

SEQ ID NO: 7 - DNA, 28 bp, Artificial Sequence
  Description of Artificial Sequence Synthetic construct
gcgactgatggcgagtgcaaagcgtggc

SEQ ID NO: 8 - DNA, 28 bp, Artificial Sequence
  Description of Artificial Sequence Synthetic construct
ttataggactgccacgctttggacgcgg

SEQ ID NO: 9 - DNA, 26 bp, Artificial Sequence
  Description of Artificial Sequence Synthetic construct
ttataggactccgcgtcctcaccggt

SEQ ID NO: 10 - DNA, 26 bp, Artificial Sequence
  Description of Artificial Sequence Synthetic construct
catcagtcgcaccggtgacgttccgc

SEQ ID NO: 11 - DNA, 28 bp, Artificial Sequence
  Description of Artificial Sequence Synthetic construct
ggcaccaggcgcggaacgaagcactcgc

SEQ ID NO: 12 - DNA, 28 bp, Artificial Sequence
  Description of Artificial Sequence Synthetic construct
gcgactgatggcgagtgcaaagcgtggc

SEQ ID NO: 13 - DNA, 28 bp, Artificial Sequence
  Description of Artificial Sequence Synthetic construct
agtcctataagccacgctttggacgcgg

SEQ ID NO: 14 - DNA, 26 bp, Artificial Sequence
  Description of Artificial Sequence Synthetic construct
agtcctataaccgcgtcctcaccggt

SEQ ID NO: 15 - DNA, 26 bp, Artificial Sequence
  Description of Artificial Sequence Synthetic construct
catcagtcgcaccggtgacgttccgc